SEQUENCE LISTING (9 sequences)

SEQ ID NO: 1 - protein, 266 aa, Artificial Sequence
  Domain Ia of Pseudomonas aeruginosa exotoxin A
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKP

SEQ ID NO: 2 - protein, 153 aa, Artificial Sequence
  Domain II Pseudomonas aeruginosa exotoxin A
TVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNY

SEQ ID NO: 3 - protein, 35 aa, Artificial Sequence
  V3 loop of HIV-1 gp120 protein
CTRPNYNKRKRIHIGPGRAFYTTKNIIGTIRQAHC

SEQ ID NO: 4 - protein, 206 aa, Artificial Sequence
  nef protein of HIV-1 or HIV-2
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 5 - protein, 35 aa, Artificial Sequence
  V3 loop of Thai-E strain of HIV
CTRPSNNTRTSITIGPGQVFYRTGDIIGDIRKAYC

SEQ ID NO: 6 - protein, 5 aa, Artificial Sequence
  ER retention signal
REDLK

SEQ ID NO: 7 - protein, 4 aa, Artificial Sequence
  ER retention signal
REDL

SEQ ID NO: 8 - protein, 4 aa, Artificial Sequence
  ER retention signal
KDEL

SEQ ID NO: 9 - DNA, 1839 bp, Artificial Sequence
  Polynucleotide that encodes Pseudomonas aeruginosa exotoxin A
gccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaag

(19) United States
(12) Patent Application Publication
(10) Pub. No.: US 2009/0155297 A1
(43) Pub. Date: Jun. 18, 2009

(54) Methods and Compositions for Inducing an Immune Response Against Multiple Antigens

(75) Inventor: Randall J. Mrsny, Los Altos Hills, CA (US)

Correspondence Address: JONES DAY, 222 EAST 41ST ST, NEW YORK, NY, 10017, US

(73) Assignee: TRINITY BIOSYSTEMS, INC., Menlo Park, CA (US)

(21) Appl. No.: 11/664,786

(22) PCT Filed: Oct. 4, 2005
(86) PCT No.: PCT/US05/35804

§ 371 Date: Oct. 21, 2008

Related U.S. Application Data
(60) Provisional application No. 60/616,116, filed on Oct. 4, 2004.

(51) Int. Cl. A61K 39/00; C07K 14/21; C07H 21/04; C12N 15/63

(52) U.S. Cl. 424/192.1; 530/350; 536/23.4; 435/320.1

(57) ABSTRACT

Methods and compositions for inducing an immune response against multiple antigens are provided herein. In one aspect, the invention provides a chimeric immunogen, comprising a receptor binding domain, a translocation domain, and more than one non-contiguous heterologous antigen. In other aspects, the invention provides nucleic acids encoding chimeric immunogens of the invention, kits comprising chimeric immunogens of the invention, cells expressing chimeric immunogens of the invention, and methods or using chimeric immunogens of the invention.

| | |
|---|---|
| 1 | mggkwskssv igwptvrerm rraepaadrv |
| | gaasrdlekh gaitssntaa tnaacawlea |
| 61 | qeeeevgfpv tpqvplrpmt ykaavdlshf |
| | lkekgglegl ihsqrrqdil dlwiyhtqgy |
| 121 | fpdwqnytpg pgvrypltfg wcyklvpvep |
| | dkieeankge ntsllhpvsl hgmddperev |
| 181 | lewrfdsrla fhhvarelhp eyfknc |

This application is entitled to and claims benefit of U.S. Provisional Application No. 60/616,116, filed Oct. 4, 2004, which is hereby incorporated by reference in its entirety.

### 1. FIELD OF THE INVENTION

The present invention relates, in part, to methods and compositions for inducing an immune response against two or more non-contiguous antigens. The methods and compositions rely, in part, on administering a chimeric immunogen comprising the two or more non-contiguous antigens to a subject to be immunized.

### 2. BACKGROUND

Immunization against bacterial or viral infection has greatly contributed to relief from infectious disease. Generally, immunization relies on administering an inactivated or attenuated pathogen to the subject to be immunized. For example, hepatitis B vaccines can be made by inactivating viral particles with formaldehyde, while some polio vaccines consist of attenuated polio strains that cannot mount a full-scale infection. In either case, the subject's immune system is stimulated to mount a protective immune response by interacting with the inactivated or attenuated pathogen. See, e.g., Kuby, 1997, Immunology W.H. Freeman and Company, New York.

This approach has proved successful for immunizing against a number of pathogens. Indeed, many afflictions that plagued mankind for recorded history have been essentially eliminated by immunization with attenuated or inactivated pathogens. See id. Nonetheless, this approach is not effective to immunize against infection by many pathogens that continue to pose significant public health problems. In particular, no vaccine presently exists that has been approved for immunization against infection by viruses such as HIV or HCV, or by bacteria such as Pseudomonas spp., or Chlamydia spp. The absence of such vaccines presents significant public health problems.

Previous efforts have been made to immunize against such pathogens using inactivated or attenuated versions of the pathogens have not been successful. See, e.g., Niedrig et al., 1993, Vaccine 11:67-74. Moreover, recombinant strategies for immunizing against these pathogens have not yet resulted in an approved vaccine, though attempts to design such vaccines are legion. See, e.g., U.S. Pat. Nos. 6,692,955, 6,544,780, 6,130,082, and 5,985,609.

One strategy for producing recombinant vaccines is presented in International Patent Publication No. WO 99/02713. The recombinant vaccines described therein generally comprise a chimeric immunogen that has functional domains corresponding to the domains of Pseudomonas aeruginosa exotoxin A and a single non-native epitope. The chimeric immunogens can elicit a humoral, cell-mediated, or secretory immune response depending on the configuration of the immunogen and/or the method of administration of the immunogen to a subject in whom the immune response is induced.

### 3. SUMMARY OF THE INVENTION

The present invention provides chimeric immunogens that comprise two or more non-contiguous heterologous antigens and can elicit humoral, cell-mediated and/or secretory immune responses against one or more of the heterologous antigens. By including two or more non-contiguous heterologous antigens in the chimeric immunogens, effective and potent immune responses can be mounted against one or more of the antigens. The chimeric immunogens are useful, for example, in compositions that can reduce or prevent infection by organisms for which conventional vaccines are not practical, by organisms that have more than one antigen against which an immune response can be raised, by organisms that have antigens that are genetically diverse and thus vary from strain to strain, by multiple organisms, or that can reduce or prevent growth of a particular cell or cells, e.g., cancer cells.

Accordingly, in certain aspects, the invention provides a chimeric immunogen for inducing an immune response, said chimeric immunogen comprising a cell surface receptor binding domain, an exotoxin translocation domain, and more than one non-contiguous antigen. In certain embodiments, the cell surface receptor binding domain is selected from the group consisting of domain Ia of Pseudomonas exotoxin A; a receptor binding domain from cholera toxin, diptheria toxin, shiga toxin, or shiga-like toxin; a monoclonal antibody, a polyclonal antibody, or a single-chain antibody; TGFα, TGFβ, EGF, PDGF, IGF, or FGF; IL-1, IL-2, IL-3, or IL-6; and MIP-1α, MIP-1b, MCAF, or IL-8. In a preferred embodiment, the cell surface receptor binding domain is domain Ia of Pseudomonas aeruginosa exotoxin A. In certain embodiments, the domain Ia of Pseudomonas aeruginosa exotoxin A has an amino acid sequence that is SEQ ID NO.: 1.

In certain embodiments, the exotoxin translocation domain is selected from the group consisting of domain II of Pseudomonas aeruginosa exotoxin A, diptheria toxin, pertussis toxin, cholera toxin, heat-labile E. coli enterotoxin, shiga toxin, and shiga-like toxin. In a preferred embodiment, the exotoxin translocation domain is domain II of Pseudomonas aeruginosa exotoxin A. In certain embodiments, the domain II of Pseudomonas aeruginosa exotoxin A has an amino acid sequence that is SEQ ID NO.: 2.

In certain embodiments, at least one of the antigens is connected with the exotoxin translocation domain or the cell surface receptor binding domain with a covalent bond. In certain embodiments, the covalent bond is a peptide bond.

In certain embodiments, at least one of the antigens is from a pathogen. In certain embodiments, at least one of the antigens is from a cancer cell. In certain embodiments, at least one antigen is from a pathogen and at least one antigen is from a cancer cell.

In certain embodiments, at least two of the antigens are from the same antigenic molecule. In other embodiments, none of the antigens is from the same antigenic molecule.

In certain embodiments, at least one of the antigens is a B cell antigen. In certain embodiments, at least one of the antigens is a T cell antigen. In certain embodiments, at least one of the antigens is a B cell antigen and at least one of the antigens is a T cell antigen.

In certain embodiments, at least one of the antigens is a peptide or polypeptide antigen. In certain embodiments, at least two of the antigens are from the same peptide or polypeptide. In certain embodiments, none of the antigens is from the same peptide or polypeptide. In certain embodiments, all of the antigens are peptide or polypeptide antigens. In certain embodiments, at least two of the antigens are from the same peptide or polypeptide. In certain embodiments, none of the antigens is from the same peptide or polypeptide.

In certain embodiments, the chimeric immunogen further comprises at least a portion of domain Ib of Pseudomonas aeruginosa exotoxin A and at least one of the antigens is inserted into the domain Ib. In certain embodiments, the antigen replaces one or more amino acids of domain Ib. In certain embodiments, the antigen that is inserted into domain Ib of Pseudomonas aeruginosa exotoxin A comprises an antigen of the V3 loop of HIV-1 gp120 protein. In certain embodiments, the antigen is the V3 loop of HIV-1 gp120 protein. In certain embodiments, the V3 loop of HIV-1 gp120 protein has an amino acid sequence that is SEQ ID NO.:3.

In certain embodiments, the polypeptide further comprises at least a portion of an enzymatically inactive domain III of Pseudomonas aeruginosa exotoxin A and at least one of the antigens is inserted into or replaces a portion of domain Ill. In certain embodiments, the antigen inserted into domain III is a T cell antigen. In certain embodiments, the Domain III of Pseudomonas aeruginosa exotoxin A comprises an endoplasmic reticulum retention signal. In certain embodiments, the antigen that is inserted into the portion of an enzymatically inactive Domain III of Pseudomonas aeruginosa exotoxin A comprises an antigen od nef protein of HIV, e.g., HIV-1 or HIV-2. In certain embodiments, the nef protein of HIV has an amino acid sequence that is SEQ ID NO.:4. In certain embodiments, the chimeric immunogen comprises at least a portion of an enzymatically inactive Domain III of Pseudomonas aeruginosa exotoxin A, at least one of the antigens is inserted into Domain III, wherein the antigen that is inserted into the portion of Domain III of Pseudomonas aeruginosa exotoxin A is nef protein of HIV-1. In certain embodiments, the V3 loop of HIV-1 gp120 protein has an amino acid sequence that is SEQ ID NO.:3 and the nef protein of HIV-1 has an amino acid sequence that is SEQ ID NO.:4.

In another aspect, the invention provides a polynucleotide encoding a chimeric immunogen of the invention.

In yet another aspect, the invention provides an expression vector that comprises a polynucleotide encoding a chimeric immunogen of the invention operably linked to an expression regulatory sequence, e.g., a promoter. In certain embodiments, the promoter is a eukaryotic promoter. In certain embodiments, the promoter is a prokaryotic promoter. In certain embodiments, the promoter is an inducible promoter. In certain embodiments, the expression vector further comprises a secretion signal that directs secretion of a polypeptide expressed from the expression vector from the cell in which the polypeptide is expressed.

In still another aspect, the invention provides a transformed or transfected cell that comprises an expression vector encoding a chimeric immunogen of the invention.

In yet another aspect, the invention provides a composition comprising a chimeric immunogen of the invention. In certain embodiments, the composition further comprises a pharmaceutically acceptable diluent, excipient, vehicle, or carrier. In certain embodiments, the composition is formulated for nasal or oral administration.

In yet another aspect, the invention provides a method for inducing an immune response in a subject, the method comprising contacting an apical epithelial membrane of said subject with an effective amount of a chimeric immunogen of the invention. In certain embodiments, the immune response is a humoral immune response. In certain embodiments, the immune response is a secretory immune response. In certain embodiments, the immune response is a cell-mediated immune response.

In certain embodiments, the chimeric immunogen is administered in the form of a pharmaceutical composition, wherein the pharmaceutical composition comprises said chimeric immunogen and a pharmaceutically acceptable diluent, adjuvant, excipient, vehicle, or carrier. In certain embodiments, the pharmaceutical composition is formulated for nasal or oral administration.

In certain embodiments, the chimeric immunogen is administered to said subject nasally or orally. In certain embodiments, the chimeric immunogen is administered to a mammal. In certain embodiments, the chimeric immunogen is administered to a rodent, lagomorph or primate. In a preferred embodiment, the chimeric immunogen is administered to a human.

### 4. BRIEF DESCRIPTION OF THE FIGURES

FIG. 1 presents the amino acid sequence of the HIV nef protein.

FIG. 2 presents a representative amino acid sequence of Pseudomonas aeruginosa exotoxin A.

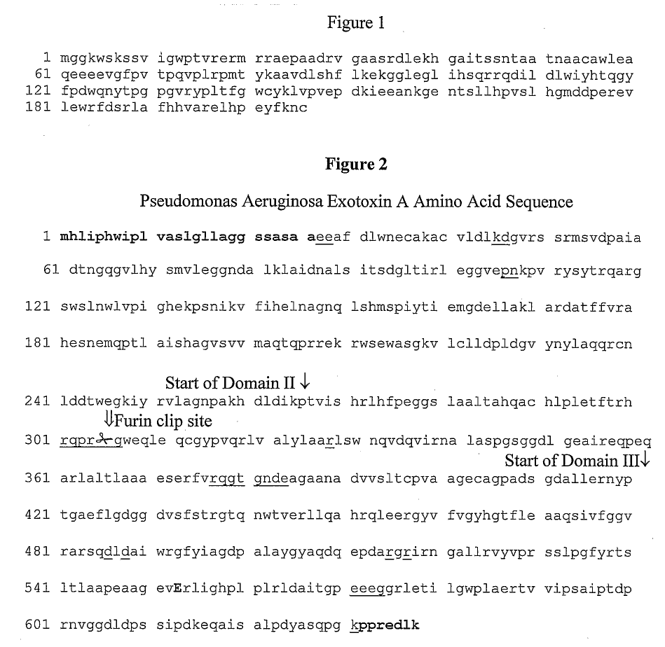

### 5. DETAILED DESCRIPTION OF THE INVENTION

### 5.1. Definitions

Unless defined otherwise, all technical and scientific terms used herein have the meaning commonly understood by a person skilled in the art to which this invention belongs. As used herein, the following terms have the meanings ascribed to them unless specified otherwise.

A “ligand” is a compound that specifically binds to a target molecule. Exemplary ligands include, but are not limited to, an antibody, a cytokine, a substrate, a signaling molecule, and the like.

A “receptor” is compound that specifically binds to a ligand.

A ligand or a receptor (e.g., an antibody) “specifically binds to” or “is specifically immunoreactive with” another molecule when the ligand or receptor functions in a binding reaction that indicates the presence of the molecule in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to another polynucleotide comprising a complementary sequence and an antibody specifically binds under immunoassay conditions to an antigen bearing an epitope used to induce the antibody.

“Immunoassay” refers to a method of detecting an analyte in a sample involving contacting the sample with an antibody that specifically binds to the analyte and detecting binding between the antibody and the analyte. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity. In one example, an antibody that binds a particular antigen with an affinity (Kₘ) of about 10 μM specifically binds the antigen.

“Vaccine” refers to an agent or composition containing an agent effective to confer a prophylactic or therapeutic degree of immunity on an organism while causing only very low levels of morbidity or mortality. Methods of making vaccines are, of course, useful in the study of the immune system and in preventing and treating animal or human disease.

An “immune response” refers to one or more biological activities mediated by cells of the immune system in a subject. Such biological activities include, but are not limited to, production of antibodies; activation and proliferation of immune cells, such as, e.g., B cells, T cells, macrophages, leukocytes, lymphocytes, etc.; release of messenger molecules, such as cytokines, chemokines, interleukins, tumor necrosis factors, growth factors, etc.; and the like. An immune response is typically mounted when a cell of the immune system encounters non-self antigen that is recognized by a receptor present on the surface of the immune cell. The immune response preferably protects the subject to some degree against infection by a pathogen that bears the antigen against which the immune response is mounted.

An “immunogen” is a molecule or combination of molecules that can induce an immune response in a subject when the immunogen is administered to the subject.

An “antigen” is a molecule, e.g., a peptide, polypeptide, carbohydrate, lipid, nucleic acid, small organic molecule, or a combination thereof, against which an immune response is induced when a molecule comprising the antigen is administered to a subject. At a minimum, an antigen comprises at least one epitope. An “antigen” is also a molecule, e.g., a peptide, polypeptide, carbohydrate, lipid, nucleic acid, or a combination thereof, that can potentiate an immune response induced against another antigen when both antigens are administered to a subject, either sequentially or simultaneously. Further, as used herein, an “antigen” is not Pseudomonas aeruginosa exotoxin A, or any portion thereof, or a receptor binding domain or translocation domain, or a portion thereof, as defined herein.

“Non-contiguous,” as used herein with respect to two or more antigens, means that the two or more antigens are not directly linked to each other by a covalent bond in the native molecule from which the two or more antigens are obtained. For example, in the case of two peptide antigens, the two peptide antigens are separated by, for example, a linker or at least one amino acid that is not part of either antigen.

“Immunizing” refers to administering an immunogen to a subject.

An “immunogenic amount” of a compound is an amount of the compound effective to elicit an immune response in a subject.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences, or a peptide that joins two other peptides, or a specific binding pair such as, e.g., streptavidin and biotin.

“Pharmaceutical composition” refers to a composition suitable for pharmaceutical use in a mammal. A pharmaceutical composition comprises a pharmacologically effective amount of an active agent and a pharmaceutically acceptable carrier. “Effective amount” or “pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 20th Ed. 2000, Mack Publishing Co., Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

Preferred pharmaceutical carriers depend upon the intended mode of administration of the active agent. Typical modes of administration include enteral (e.g., oral, intranasal, rectal, or vaginal) or parenteral (e.g., subcutaneous, intramuscular, intravenous or intraperitoneal injection; or topical, transdermal, or transmucosal administration).

“Small organic molecule” refers to organic molecules of a size comparable to those organic molecules generally used in pharmaceuticals. The term excludes organic biopolymers (e.g., proteins, nucleic acids, etc.). Preferred small organic molecules range in size up to about 5000 Da, up to about 2000 Da, or up to about 1000 Da.

A “subject” of diagnosis, treatment, or administration is a human or non-human animal, including a mammal, such as a rodent (e.g., a mouse or rat), a lagomorph (e.g., a rabbit), or a primate. A subject of diagnosis, treatment, or administration is preferably a primate, and more preferably a human.

An immune response may be “elicited,” “induced,” or “induced against” a particular antigen. Each of these terms is intended to be synonymous as used herein and refers to the ability of the chimeric immunogen to generate an immune response upon administration to a subject.

“Treatment” refers to prophylactic treatment or therapeutic treatment. A “prophylactic” treatment is a treatment administered to a subject who does not exhibit signs of a disease or exhibits only early signs for the purpose of decreasing the risk of developing pathology. A “therapeutic” treatment is a treatment administered to a subject who exhibits signs of pathology for the purpose of diminishing, slowing the progression, eliminating, or halting those signs.

“Pseudomonas exotoxin A” or “PE” is secreted by Pseudomonas aeruginosa as a 67 kD protein composed of three prominent globular domains (Ia, II, and III) and one small subdomain (Ib) that connects domains II and III. See A. S. Allured et al., 1986, Proc. Natl. Acad. Sci. 83:1320-1324, and FIG. 2, which presents the amino acid sequence of native PE. Without intending to be bound to any particular theory or mechanism of action, domain Ia of PE is believed to mediate cell binding because domain Ia specifically binds to the low density lipoprotein receptor-related protein (“LRP”), also known as the α2-macroglobulin receptor (“α2-MR”) and CD-91. See M. Z. Kounnas et al., 1992, J. Biol. Chem. 267:12420-23. Domain Ia spans amino acids 1-252. Domain III of PE is believed to mediate translocation to the interior of a cell following binding of domain Ia to the α2-MR. Domain II spans amino acids 253-364. Domain Ib has no apparent function and spans amino acids 365-399. Domain III mediates cytotoxicity of PE and includes an endoplasmic reticulum retention sequence. PE cytotoxicity is believed to result from ADP ribosylation of elongation factor 2, which inactivates protein synthesis. Domain III spans amino acids 400-613 of PE. Deleting amino acid E553 (“ΔE553”) from domain III eliminates EF2 ADP ribosylation activity and detoxifies PE. PE having the mutation ΔE553 is referred to herein as “PEΔE553.” Genetically modified forms of PE are described in, e.g., U.S. Pat. Nos. 5,602,095; 5,512,658 and 5,458,878. Pseudomonas exotoxin, as used herein, also includes genetically modified, allelic, and chemically inactivated forms of PE within this definition. See, e.g., Vasil et al., 1986, Infect. Immunol. 52:538-48. Further, reference to the various domains of PE is made herein to the reference PE sequence presented as FIG. 2. However, one or more domain from modified PE, e.g., genetically or chemically modified PE, or a portion of such domains, can also be used in the chimeric immunogens of the invention so long as the domains retain functional activity. One of skill in the art can readily identify such domains of such modified PE based on, for example, homology to the PE sequence exemplified in FIG. 2 and test for functional activity using, for example, the assays described below.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions. Thus, the polynucleotide whose sequence 5′-TATAC-3′ is complementary to a polynucleotide whose sequence is 5′-GTATA-3′.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and RNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Amplification” refers to any means by which a polynucleotide sequence is copied and thus expanded into a larger number of polynucleotide molecules, e.g., by reverse transcription, polymerase chain reaction, ligase chain reaction, and the like.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, NY; Sambrook et al., 2001, Molecular Cloning: A Laboratory Manual, Cold Spring Harbor Laboratory, 3^{rd} ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

One example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. Conventional notation is used herein to portray polypeptide sequences; the beginning of a polypeptide sequence is the amino-terminus, while the end of a polypeptide sequence is the carboxyl-terminus.

The term “protein” typically refers to large polypeptides, for example, polypeptides comprising more than about 50 amino acids. The term “protein” can also refer to dimers, trimers, and multimers that comprise more than one polypeptide.

The term “peptide” typically refers to short polypeptides, for example, polypeptides comprising about 50 or less amino acids, e.g., about five to about thirty amino acids.

“Conservative substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W).

### 5.2. Chimeric Immunogens

Generally, the chimeric immunogens of the present invention are polypeptides that comprise structural domains corresponding to domains Ia and II of PE. The chimeric immunogens can optionally comprise structural domains corresponding to the other domains of PE, domains Ib and III. These structural domains perform certain functions, including, but not limited to, cell recognition, translocation and endoplasmic reticulum retention, that correspond to the functions of the domains of PE. By including or omitting the optional domains of PE, the character of the induced immune response can be modulated, as described below.

In addition to the portions of the molecule that correspond to PE functional domains, the chimeric immunogens of this invention further comprise two or more non-contiguous heterologous antigens. The heterologous antigens can be introduced into domain Ib or domain III of PE, or the heterologous antigens can be introduced into any other portion of the molecule that does not disrupt a cell-binding or translocation activity. An immune response specific for one or more of the heterologous antigens is elicited upon administration of the chimeric immunogen to a subject.

Accordingly, the chimeric immunogens of the invention generally comprise the following structural elements, each element imparting particular functions to the chimeric immunogen: (1) a “receptor binding domain” that functions as a ligand for a cell surface receptor and that mediates binding of the chimeric immunogen to a cell; (2) a “translocation domain” that mediates translocation of the chimeric immunogen from the exterior of the cell to the interior of the cell; (3) the two or more heterologous antigens; and, optionally, (4) an “endoplasmic reticulum (“ER”) retention domain” that translocates the chimeric immunogen from the endosome to the endoplasmic reticulum, from which it enters the cytosol. The chimeric immunogen can still induce an immune response in the absence of the ER retention domain, though this absence changes the nature of the induced immune response, as described below.

The domains of the chimeric immunogens other than the heterologous antigens can be present in the order set forth above, i.e., domain Ia is closest to the N-terminus, then the translocation domain, then the ER retention domain. In fact, this arrangement is preferred. However, the domains of the chimeric immunogen can be in any order as long as the domains retain their functional activities. Several representative assays to test such functional activities are set forth below.

Such chimeric immunogens offer several advantages over conventional immunogens. To begin with, certain embodiments of the chimeric immunogens can be constructed and expressed in recombinant systems. These systems eliminate any requirement to crosslink the heterologous antigens to a carrier protein. Recombinant technology also allows one to make a chimeric immunogen having one or more insertion sites designed for introduction of any desired heterologous antigens. Such insertion sites allow the skilled artisan to quickly and easily produce chimeric immunogens that comprise either known variants of heterologous antigens or emerging variants of evolving heterologous antigens.

Further, the chimeric immunogens can be engineered to alter the function of their domains in order to tailor the activity of the immunogen to its intended use. For example, by selecting the appropriate receptor binding domain, the skilled artisan can target the chimeric immunogen to bind to a desired cell or cell line.

In addition, because certain embodiments of the chimeric immunogens include a constrained cysteine-cysteine loop, heterologous antigens that are so constrained in nature can be presented in native or near-native conformation. By doing so, the induced immune response is specific for antigen in its native conformation, and can more effectively protect the subject from infection by the pathogen. For example, a helix-turn-helix motif can be observed in peptides constrained by a disulfide bond, but not in linear peptides. See Ogata et al., 1990, Biol. Chem. 265:20678-85.

Moreover, the chimeric immunogens can be used to elicit a humoral, a cell-mediated and/or a secretory immune response. Depending on the pathway by which the chimeric immunogen is processed in an antigen-presenting cell, the chimeric immunogen can induce an immune response mediated by either class I or class II MHC. See Becerrra et al., 2003, Surgery 133:404-410 and Lippolis et al., 2000, Cell. Immunol. 203:75-83. Further, if the PE chimeras are administered to a mucosal surface of the subject, a secretory immune response involving IgA can be induced. See, e.g., Mrsny et al., 1999, Vaccine 17:1425-1433 and Mrsny et al., 2002, Drug Discovery Today 7:247-258.

The chimeric immunogens of the invention can also be used to elicit a protective immune response without using attenuated or inactivated pathogens. The inactivation or attenuation of such pathogens can sometimes be incomplete, or the pathogen can revert to be fully infectious, leading to infection by the pathogen upon administration of the vaccine. For example, administration of attenuated polio vaccine actually results in paralytic polio in about 1 in 4 million subjects receiving the vaccine. See Kuby, 1997, Immunology Ch. 18, W.H. Freeman and Company, New York.

Further, including two or more heterologous antigens in the chimeric immunogens provides additional flexibility in design and construction of the immunogen and additional options in the immune responses that can be induced. For example, a B cell antigen and a helper T cell antigen can be delivered to the immune system in the same construct, potentiating the humoral immune response against the B cell antigen. In another example, cytotoxic T cell antigens from two different molecules from a pathogen can be delivered to the immune system in one construct, resulting in an immune response of broader specificity than if only one of the antigens were administered. Other advantages of chimeric immunogens that comprise two or more antigens will be apparent to those of skill in the art.

5.2.1. Receptor Binding Domain

The chimeric immunogens of the invention generally comprise a receptor binding domain. The receptor binding domain can be any receptor binding domain that binds to a cell surface receptor, without limitation. Such receptor binding domains are well-known to those of skill in the art. Preferably, the receptor binding domain binds specifically to the cell surface receptor, e.g., binds to the cell surface receptor with an affinity that is greater, preferably at least an order of magnitude greater, than the affinity of the receptor binding domain for unrelated ligands. In certain embodiments, the receptor binding domain binds to the cell surface receptor with binding constant (Kₘ) of at least about 1 mM, 10 μM, 1 μM, 100 nM, 10 nM, or 1 nM. The receptor binding domain should bind to the cell surface receptor with sufficient affinity to allow endocytosis of the chimeric immunogen. Representative assays that can routinely be used by the skilled artisan to assess binding of the receptor binding domain to a cell surface receptor are described below.

The receptor binding domain is generally present at the N-terminal end of the chimeric immunogen, or, alternative, is at least generally amino to the heterologous antigens, translocation domain, and optional ER retention domain.

In certain embodiments, the receptor binding domain can comprise a polypeptide, a peptide, a protein, a lipid, a carbohydrate, or a small organic molecule, or a combination thereof. Examples of each of these molecules that bind to cell surface receptors are well known to those of skill in the art. Suitable peptides, polypeptides, or proteins include, but are not limited to, bacterial toxin receptor binding domains, such as the receptor binding domains from PE, cholera toxin, diptheria toxin, shiga toxin, shiga-like toxin, etc.; antibodies, including monoclonal, polyclonal, and single-chain antibodies, or derivatives thereof, growth factors, such as TGFα, TGFβ, EGF, PDGF, IGF, FGF, etc.; cytokines, such as IL-1, IL-2, IL-3, IL-6, etc; chemokines, such as MIP-1a, MIP-1b, MCAF, IL-8, etc.; and other ligands, such as CD4, cell adhesion molecules from the immunoglobulin superfamily, integrins, ligands specific for the IgA receptor, etc. See, e.g., Pastan et al, 1992, Annu. Rev. Biochem. 61:331-54; and U.S. Pat. Nos. 5,668,255, 5,696,237, 5,863,745, 5,965,406, 6,022,950, 6,051,405, 6,251,392, 6,440,419, and 6,488,926. The skilled artisan can routinely select the appropriate receptor binding domain based upon the expression pattern of the receptor to which the receptor binding domain binds.

Lipids suitable for receptor binding domains include, but are not limited to, lipids that themselves bind cell surface receptors, such as sphingosine-1-phosphate, lysophosphatidic acid, sphingosylphosphorylcholine, retinoic acid, etc.; lipoproteins such as apolipoprotein E, apolipoprotein A, etc., and glycolipids such as lipopolysaccharide, etc.; glycosphingolipids such as globotriaosylceramide and galabiosylceramide; and the like. Carbohydrates suitable for receptor binding domains include, but are not limited to, monosaccharides, disaccharides, and polysaccharides that comprise simple sugars such as glucose, fructose, galactose, etc.; and glycoproteins such as mucins, selectins, and the like. Suitable small organic molecules for receptor binding domains include, but are not limited to, vitamins, such as vitamin A, B₁, B₂, B₃, B₆, B₉, B₁₂, C, D, E, and K, amino acids, and other small molecules that are recognized and/or taken up by receptors present on the surface of epithelial cells.

In certain embodiments, the receptor binding domain can bind to a receptor found on an epithelial cell. In further embodiments, the receptor binding domain can bind to a receptor found on the apical membrane of an epithelial cell. In still further embodiments, the receptor binding domain can bind to a receptor found on the apical membrane of a mucosal epithelial cell. The receptor binding domain can bind to any receptor present on the apical membrane of an epithelial cell without limitation. For example, the receptor binding domain can bind to α2-MR. An example of a receptor binding domain that can bind to α2-MR is domain Ia of PE. Accordingly, in certain embodiments, the receptor binding domain is domain Ia of PE. In other embodiments, the receptor binding domain is a portion of domain Ia of PE that can bind to α2-MR.

In certain embodiments, the receptor binding domain can bind to a receptor present on an antigen presenting cell, such as, for example, a dendritic cell or a macrophage. The receptor binding domain can bind to any receptor present on an antigen presenting cell without limitation. For example, the receptor binding domain can bind to any receptor identified as present on a dendritic or other antigen presenting cell identified in Figdor, 2003, Pathol. Biol. (Paris). 51(2):61-3; Coombes et al., 2001, Immunol Lett. 3; 78(2):103-11; Shortman K et al., 1997, Ciba Found Symp. 204:130-8; discussion 138-41; Katz, 1998, Curr Opin Immunol. 1(2):213-9; and Goldsby et al., 2003, Immunology, 5th Edition W. H. Freeman & Company, New York, N.Y. In particular, the receptor binding domain can bind to α2-MR, which is also expressed on the surface of antigen presenting cells. Thus, in certain embodiments, the receptor binding domain can bind to a receptor that is present on both an epithelial cell and on an antigen presenting cell.

In certain embodiments, the chimeric immunogens of the invention comprise more than one domain that can function as a receptor binding domain. For example, the chimeric immunogen can comprise PE domain Ia in addition to another receptor binding domain.

The receptor binding domain can be attached to the remainder of the chimeric immunogen by any method or means known by one of skill in the art to be useful for attaching such molecules, without limitation. In certain embodiments, the receptor binding domain is expressed together with the remainder of the chimeric immunogen as a fusion protein. Such embodiments are particularly useful when the receptor binding domain and the remainder of the immunogen are formed from peptides or polypeptides.

In other embodiments, the receptor binding domain is connected with the remainder of the chimeric immunogen with a linker. In yet other embodiments, the receptor binding domain is connected with the remainder of the chimeric immunogen without a linker. Either of these embodiments are useful when the receptor binding domain comprises a peptide, polypeptide, protein, lipid, carbohydrate, nucleic acid, or small organic molecule.

In certain embodiments, the linker can form a covalent bond between the receptor binding domain and the remainder of the chimeric immunogen. In other embodiments, the linker can link the receptor binding domain to the remainder of the chimeric immunogen with one or more non-covalent interactions of sufficient affinity. One of skill in the art can readily recognize linkers that interact with each other with sufficient affinity to be useful in the chimeric immunogens of the invention. For example, biotin can be attached to the receptor binding domain, and streptavidin can be attached to the remainder of the molecule. In certain embodiments, the linker can directly link the receptor binding domain to the remainder of the molecule. In other embodiments, the linker itself comprises two or more molecules that associate in order to link the receptor binding domain to the remainder of the molecule. Exemplary linkers include, but are not limited to, straight or branched-chain carbon linkers, heterocyclic carbon linkers, substituted carbon linkers, unsaturated carbon linkers, aromatic carbon linkers, peptide linkers, etc.

In embodiments where a linker is used to connect the receptor binding domain to the remainder of the chimeric immunogen, the linkers can be attached to the receptor binding domain and/or the remainder of the chimeric immunogen by any means or method known by one of skill in the art without limitation. For example, the linker can be attached to the receptor binding domain and/or the remainder of the chimeric immunogen with an ether, ester, thioether, thioester, amide, imide, disulfide or other suitable moiety. The skilled artisan can select the appropriate linker and means for attaching the linker based on the physical and chemical properties of the chosen receptor binding domain and the linker. The linker can be attached to any suitable functional group on the receptor binding domain or the remainder of the molecule. For example, the linker can be attached to sulfhydryl (—S), carboxylic acid (COOH) or free amine (—NH2) groups, which are available for reaction with a suitable functional group on a linker. These groups can also be used to connect the receptor binding domain directly connected with the remainder of the molecule in the absence of a linker.

Further, the receptor binding domain and/or the remainder of the chimeric immunogen can be derivatized in order to facilitate attachment of a linker to these moieties. For example, such derivatization can be accomplished by attaching suitable derivative such as those available from Pierce Chemical Company, Rockford, Ill. Alternatively, derivatization may involve chemical treatment of the receptor binding domain and/or the remainder of the molecule. For example, glycol cleavage of the sugar moiety of a carbohydrate or glycoprotein receptor binding domain with periodate generates free aldehyde groups. These free aldehyde groups may be reacted with free amine or hydrazine groups on the remainder of the molecule in order to connect these portions of the molecule. See U.S. Pat. No. 4,671,958. In addition, the skilled artisan can generate free sulfhydryl groups on proteins to provide a reactive moiety for making a disulfide, thioether, theioester, etc. linkage. See U.S. Pat. No. 4,659,839.

Any of these methods for attaching a linker to a receptor binding domain and/or the remainder of a chimeric immunogen can also be used to connect a receptor binding domain with the remainder of the chimeric immunogen in the absence of a linker. In such embodiments, the receptor binding domain is coupled with the remainder of the immunogen using a method suitable for the particular receptor binding domain. Thus, any method suitable for connecting a protein, peptide, polypeptide, nucleic acid, carbohydrate, lipid, or small organic molecule to the remainder of the chimeric immunogen, can be used to connect the receptor binding domain to the remainder of the immunogen. In addition to the methods for attaching a linker to a receptor binding domain or the remainder of an immunogen, as described above, the receptor binding domain can be connected with the remainder of the immunogen as described in any of U.S. Pat. Nos. 6,673,905; 6,585,973; 6,596,475; 5,856,090; 5,663,312; 5,391,723; 6,171,614; 5,366,958; and 5,614,503.

In certain embodiments, the receptor binding domain can be a monoclonal antibody or antigen-binding portion of an antibody. In some of these embodiments, the chimeric immunogen is expressed as a fusion protein that comprises an immunoglobulin heavy chain from an immunoglobulin specific for a receptor on a cell to which the chimeric immunogen is intended to bind, or antigen-binding portion thereof. The light chain of the immunoglobulin, or antigen-binding portion thereof, then can be co-expressed with the chimeric immunogen, thereby forming an antigen-binding light chain-heavy chain dimer. In other embodiments, the antibody, or antigen-binding portion thereof, can be expressed and assembled separately from the remainder of the chimeric immunogen and chemically linked thereto.

5.2.2. Translocation Domain

The chimeric immunogens of the invention also comprise a translocation domain. The translocation domain can be any translocation domain known by one of skill in the art to effect translocation of chimeric proteins that have bound to a cell surface receptor from outside the cell to inside the cell, e.g., the outside of an epithelial cell, such as, for example, a polarized epithelial cell. In certain embodiments, the translocation domain is a translocation domain from PE, diptheria toxin, pertussis toxin, cholera toxin, heat-labile E. coli enterotoxin, shiga toxin, or shiga-like toxin. See, for example, U.S. Pat. Nos. 5,965,406, and 6,022,950. In preferred embodiments, the translocation domain is domain II of PE.

The translocation domain need not, though it may, comprise the entire amino acid sequence of domain II of native PE, which spans residues 253-364 of PE. For example, the translocation domain can comprise a portion of PE that spans residues 280-344 of domain II of PE. The amino acids at positions 339 and 343 appear to be necessary for translocation. See Siegall et al., 1991, Biochemistry 30:7154-59. Further, conservative or nonconservative substitutions can be made to the amino acid sequence of the translocation domain, as long as translocation activity is not substantially eliminated. A representative assay that can routinely be used by one of skill in the art to determine whether a translocation domain has translocation activity is described below.

Without intending to be limited to any particular theory or mechanism of action, the translocation domain is believed to perform at least two important functions in the chimeric immunogens of the invention. First, the translocation domain permits the trafficking of the chimeric immunogen through a polarized epithelial cell into the bloodstream after the immunogen binds to a receptor present on the apical surface of the polarized epithelial cell. This trafficking results in the release of the chimeric immunogen from the basal-lateral membrane of the polarized epithelial cell. Second, the translocation domain facilitates endocytosis of the chimeric immunogen into an antigen presenting cell after the immunogen binds to a receptor present on the surface of the antigen presenting cell.

5.2.3. Heterologous Antigens

The chimeric immunogens of the invention also comprise two or more non-contiguous heterologous antigens. The antigens are “heterologous” because they are heterologous to at least a portion of the remainder of the immunogen; i.e., not ordinarily found in a molecule from which at least one of the other domains of the chimeric immunogen is derived.

The heterologous antigens can each be any molecule, macromolecule, combination of molecules, etc. against which an immune response is desired or which can potentiate an immune response against another antigen. Thus, the heterologous antigens can each be any peptide, polypeptide, protein, nucleic acid, lipid, carbohydrate, or small organic molecule, or any combination thereof, against which the skilled artisan wishes to induce an immune response or which can potentiate an immune response induced against another antigen. Preferably, the heterologous antigens are each an antigen that is present on a pathogen. More preferably, the heterologous antigens are each an antigen that, when administered to a subject as part of a chimeric immunogen, results in an immune response against at least one of the heterologous antigens that protects the subject from infection by a pathogen from which at least one of the heterologous antigens are derived. In certain embodiments, the chimeric immunogen comprises more than one copy of a particular heterologous antigen.

The heterologous antigens can each be attached to the remainder of the chimeric immunogen by any method known by one of skill in the art without limitation. In certain, embodiments, the heterologous antigens are each expressed together with the remainder of the chimeric immunogen as a fusion protein. In such embodiments, the heterologous antigens can each be inserted into any portion of the chimeric immunogen, so long as the receptor binding domain, the translocation domain, and the optional ER retention signal domain retain their activities, and the immune response induced against at least one of the heterologous antigens retains specificity. Methods for assessing the specificity of the immune response against one or more of the heterologous antigens are extensively described below. The heterologous antigens are each preferably inserted into, or replace, the Ib loop of PE, into the ER retention domain, e.g., domain III, or attached to or near the C-terminal end of the translocation domain.

In native PE, the Ib loop (domain Ib) spans amino acids 365 to 399, and is structurally characterized by a disulfide bond between two cysteines at positions 372 and 379. This portion of PE is not essential for any known activity of PE, including cell binding, translocation, ER retention or ADP ribosylation activity. Accordingly, domain Ib can be deleted entirely, or modified to contain one or more heterologous antigens.

Thus, in certain embodiments, one or more of the heterologous antigens can be inserted into or replace all or a portion of domain Ib. If desirable, the heterologous antigen can be inserted into domain Ib wherein the cysteines at positions 372 and 379 are not crosslinked. This can be accomplished by reducing the disulfide linkage between the cysteines, by deleting the cysteines entirely from the Ib domain, by mutating at least one of the cysteines to other residues, such as, for example, serine, or by other similar techniques. Alternatively, one or more of the heterologous antigens can be inserted into the Ib loop between the cysteines at positions 372 and 379. In such embodiments, the disulfide linkage between the cysteines can be used to constrain the heterologous antigen(s) inserted between the cysteines.

This arrangement offers several advantages. The chimeric immunogens can be used in this manner to present one or more heterologous antigens that naturally comprise a cysteine-cysteine disulfide bond in native or near-native conformation. Further, without intending to be bound by any particular theory or mechanism of action, it is believed that charged amino acid residues in the native Ib domain result in a hydrophilic structure that protrudes from the molecule and into the solvent. Thus, inserting one or more of the heterologous antigens into the Ib loop gives immune system components unfettered access to the antigen, resulting in more effective antigen presentation. Such access is particularly useful when one or more of the heterologous antigens is a B cell antigen for inducing a humoral immune responses. Further, changes, including mutations or insertions, to domain Ib do not appear to affect activity of the other PE domains. Accordingly, although native Ib domain has only six amino acids between the cysteine residues, much longer sequences can be inserted into the loop without disrupting the other functions of the chimeric immunogen.

In other embodiments, one or more of the heterologous antigens can be inserted into the optional ER retention domain of the chimeric immunogen. Without intending to be bound to any particular theory or mechanism of action, it is believed that the nature of the immune response against an antigen inserted into the ER retention domain varies depending on the degree of separation between the antigen and the ER retention signal. In particular, the degree to which a heterologous antigen is processed by the Class I or II MHC pathways can vary depending on this degree of separation. By placing one or more of the heterologous antigens close to the ER retention signal, e.g., inserting one or more of the heterologous antigens into the ER retention domain of the chimeric immunogen near the ER retention signal, more of the heterologous antigen(s) so inserted can be directed into the Class I MHC processing pathway, thereby inducing a cellular immune response against the antigen(s). Conversely, when one or more of the heterologous antigens is further from the ER retention signal, more of the antigen(s) is directed into the Class II MHC processing pathway, thereby facilitating induction of a humoral immune response. If the immune response is intended to be primarily humoral, with essentially no Class I MHC cell mediated response, the ER retention domain can be deleted entirely, and the heterologous antigens attached to the immunogen in another location, such as, for example, to the C terminus of the translocation domain. Thus, by controlling the spatial relationship between one or more of the heterologous antigen and the ER retention signal, the skilled artisan can modulate the immune response that is induced against the heterologous antigens.

In embodiments where the heterologous antigens are each expressed together with another portion of the chimeric immunogen as a fusion protein, the heterologous antigens can be can be inserted into the chimeric immunogen by any method known to one of skill in the art without limitation. For example, amino acids corresponding to the heterologous antigens can be inserted directly into the chimeric immunogen, with or without deletion of native amino acid sequences. In certain embodiments, all or part of the Ib domain of PE can be deleted and replaced with one or more of the heterologous antigens. In certain embodiments, the cysteine residues of the Ib loop are deleted so that the one or more heterologous antigens remains unconstrained. In other embodiments, the cysteine residues of the Ib loop are linked with a disulfide bond and constrain the one or more heterologous antigens.

In embodiments where one or more of the heterologous antigens is not expressed together with the remainder of the chimeric immunogen as a fusion protein, the heterologous antigen(s) can be connected with the remainder of the chimeric immunogen by any suitable method known by one of skill in the art, without limitation. More specifically, the exemplary methods described above for connecting a receptor binding domain to the remainder of the molecule are equally applicable for connecting the heterologous antigen(s) to the remainder of the molecule.

In certain embodiments, the chimeric immunogen comprises two, three, four, five, six, seven, eight, nine, ten, eleven, twelve, thirteen, fourteen, fifteen, sixteen, seventeen, eighteen, nineteen, twenty, twenty-five, thirty, or more heterologous antigens. In certain embodiments, the chimeric immunogen comprises two to about five antigens, two to about eight antigens, two to about ten antigens, two to about fifteen antigens, two to about twenty antigens, two to about thirty antigens, about five to about eight antigens, about five to about ten antigens, about five to about fifteen antigens, about five to about twenty antigens, about five to about thirty antigens, about eight to about ten antigens, about eight to about fifteen antigens, about eight to about twenty antigens, about eight to about thirty antigens, about ten to about fifteen antigens, about ten to about twenty antigens, about ten to about thirty antigens, about fifteen to about twenty antigens, about fifteen to about thirty antigens, or about twenty to about thirty antigens.

In certain embodiments, one or more of the heterologous antigens can be a peptide, polypeptide, or protein. The heterologous antigen(s) can be any peptide, polypeptide, or protein against which an immune response is desired to be induced. In certain embodiments, one or more of the heterologous antigens is a peptide that comprises about 5, about 8, about 10, about 12, about 15, about 17, about 20, about 25, about 30, about 40, about 50, or about 60, about 70, about 80, about 90, about 100, about 200, about 400, about 600, about 800, or about 1000 amino acids.

In certain embodiments, all of the heterologous antigens present in the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, about 1000, about 1200, about 1400, about 1600, about 1800, or about 2000 amino acids.

In certain embodiments, all of the heterologous antigens present in a particular domain of the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, about 1000, about 1200, about 1400, about 1600, about 1800, or about 2000 amino acids.

In certain embodiments, one or more of the heterologous antigens is a carbohydrate. The heterologous antigen(s) can be any carbohydrate against which an immune response is desired to be induced. In certain embodiments, one or more of the heterologous antigens is a carbohydrate that comprises about 1, about 2, about 3, about 4, about 5, about 8, about 10, about 12, about 15, about 17, about 20, about 25, about 30, about 40, about 50, or about 60, about 70, about 80, about 90, or about 100 sugar monomers.

In certain embodiments, all of the heterologous antigens present in the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, or about 1000 sugar monomers.

In certain embodiments, all of the heterologous antigens present in a particular domain of the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, or about 1000 sugar monomers.

In other embodiments, one or more of the heterologous antigens can be a glycoprotein, or a portion thereof. The heterologous antigen(s) can be any glycoprotein, or portion of a glycoprotein, against which an immune response is desired to be induced. In certain embodiments, one or more of the heterologous antigens is a glycoprotein or glycoprotein portion that comprises about 5, about 8, about 10, about 12, about 15, about 17, about 20, about 25, about 30, about 40, about 50, or about 60, about 70, about 80, about 90, about 100, about 200, about 400, about 600, about 800, or about 1000 amino acids.

In addition to the protein component, the glycoprotein or glycoprotein portion also comprises a carbohydrate moiety. The carbohydrate moiety of the glycoprotein or glycoprotein portion comprises about 1, about 2, about 3, about 4, about 5, about 8, about 10, about 12, about 15, about 17, about 20, about 25, about 30, about 40, about 50, or about 60, about 70, about 80, about 90, or about 100 sugar monomers.

In certain embodiments, all of the heterologous antigens present in the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, about 1000, about 1200, about 1400, about 1600, about 1800, or about 2000 amino acids and about 5, about 8, about 10, about 12, about 15, about 17, about 20, about 25, about 30, about 40, about 50, or about 60, about 70, about 80, about 90, about 100, about 200, about 400, about 600, about 800, or about 1000 sugar monomers.

In certain embodiments, all of the heterologous antigens present in a particular domain of the chimeric immunogen together comprise about 5, about 8, about 10, about 12, about 15, about 17, about 20, about 25, about 30, about 40, about 50, or about 60, about 70, about 80, about 90, about 100, about 200, about 400, about 600, about 800, about 1000, about 1200, about 1400, about 1600, about 1800, or about 2000 amino acids and about 5, about 8, about 10, about 12, about 15, about 17, about 20, about 25, about 30, about 40, about 50, or about 60, about 70, about 80, about 90, about 100, about 200, about 400, about 600, about 800, or about 1000 sugar monomers.

In general, the skilled artisan may routinely select each of the two or more heterologous antigens, guided by the following discussion. One important factor in selecting the heterologous antigens is the type of immune response that is to be induced. For example, when a humoral immune response is desired, at least one of the heterologous antigens should be selected to be recognizable by a B-cell receptor and to be antigenically similar to a region of the source molecule that is available for antibody binding.

Important factors to consider when selecting a B-cell antigen include, for example, the size and conformation of the antigenic determinant to be recognized, both in the context of the chimeric immunogen and in the native molecule from which the B-cell antigen is derived; the hydrophobicity or hydrophilicity of the antigen; the topographical accessibility of the antigen in the native molecule from which the particular heterologous antigen is derived; and the flexibility or mobility of the portion of the native molecule from which the B-cell antigen is derived. See, e.g., Kuby, 1997, Immunology Chapter 4, W.H. Freeman and Company, New York. Based on these criteria, the skilled artisan can, when appropriate, select a portion of a large molecule, such as a protein, to be one of the heterologous antigens in the chimeric immunogen. If the source of the heterologous antigen cannot be effectively represented by selecting a portion of it, then the skilled artisan can select the entire molecule to be one of the heterologous antigens. Such embodiments are particularly useful in the cases of B-cell antigens that are formed by non-sequential amino acids, i.e., antigens formed by amino acids that are not adjacent in the primary structure of the source protein.

Similarly, if the skilled artisan wishes to deliver one or more heterologous antigens to activate T cells, several factors must be considered in the selection of such heterologous antigen(s). Principle among such factors is whether helper T cells or cytotoxic T cells are to be stimulated. As described below, helper T cells recognize antigen presented by Class II MHC molecules, while cytotoxic T cells recognize antigen present by Class I MHC. Accordingly, in order to selectively activate these populations, the skilled artisan should select one or more heterologous antigen to be presentable by the appropriate type of MHC. For example, the skilled artisan can select one or more of the heterologous antigens to be a peptide that is presented by Class I MHC when a response mediated by cytotoxic T cells is desired. Similarly, the skilled artisan can select one or more of the heterologous antigens to be a peptide that is presented by Class II MHC when a response mediated by helper T cells is desired.

Further, both Class I and Class II MHC exhibit significant allelic variation in studied populations. Much is known about Class I and II MHC alleles and the effects of allelic variation on antigens that can be presented by the different alleles. For example, rules for interactions between Class I MHC haplotype and antigens that can be effectively presented by these molecules are reviewed in Stevanovic, 2002, Transpl. Immunol. 10:133-136. Further guidance on selection of appropriate peptide antigens for Class I and II MHC molecules may be found in U.S. Pat. Nos. 5,824,315 and 5,747,269, and in Germain et al., 1993, Annu. Rev. Immunol. 11:403-450; Sinigaglia et al., 1994, Curr. Opin. Immunol. 6:52-56; Margalit et al., 2003, Novartis Found Symp. 254:77-101, 216-22, and 250-252; Takahashi, 2003, Comp Immunol Microbiol Infect Dis. 26:309-328; Yang, 2003, Microbes Infect. 5:39-47; and Browning et al., 1996, HLA and MHC: Genes, Molecules and Function (Davenport and Hill, eds.) A BIOS Scientific Publishers, Oxford. Empirical systems for identifying peptide antigens for presentation on Class II MHC, and that can be adapted for identifying peptide antigens for presentation on Class I MHC, are presented in U.S. Pat. Nos. 6,500,641 and 6,716,623.

Thus, for example, one or more of the heterologous antigens can be chosen from any pathogen, including, but not limited to, viruses, bacteria, fungi and protozoan or other parasites. Viral sources of heterologous antigens include, for example, HIV, herpes, influenza, polio, hepatitis B, hepatitis C, cytomegalovirus, west nile virus, hantaviurus, yellow fever virus, ebola virus, etc. Bacterial sources include, for example, Mycobacterium tuberculinum, Chlamydia spp., Salmonella spp., E. coli, Pseudomonas spp, Legionella spp., etc. Parasitic protozoan sources include, for example, Trypanosoma or Plasmodium. The chimeric immunogens are particularly useful in immunizing against pathogens that enter the subject through epithelial mucosal membranes because of the chimeric immunogens' ability to elicit a secretory immune response, as described below.

Further examples of viruses that can serve as sources of heterologous antigens include, but are not limited to: Retroviridae (e.g. human immunodeficiency viruses, such as HIV-1 (also referred to as HTLV-III, LAV or HTLV-III/LAV, or HIV-III); and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g., dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Parainyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arenaviridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Further examples of viruses that can serve as sources of heterologous antigens include, but are not limited to: both gram negative and gram positive bacteria such as Pasteurella spp., Staphylococci spp., Streptococcus spp., Escherichia coli, Pseudomonas spp., and Salmonella spp. Further specific examples of infectious bacteria include but are not limited to: Helicobacter pyloris, Borelia burgdorferi, Legionella pneumophilia, Mycobacteria spp (e.g. M. tuberculosis, M. avium, M. intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi that can serve as sources of heterologous antigens include, but are not limited to: Cryptococcus neoformans, Histoplasma capsulatum, Coccidioides immitis, Blastomyces dermatitidis, Chlamydia trachomatis, and Candida albicans.

Further examples of parasites that can serve as sources of heterologous antigens include, but are not limited to: Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms that can serve as sources of heterologous antigen have been described extensively in the literature, e.g., see C. G. A Thomas, Medical Microbiology, Bailliere Tindall, Great Britain 1983, the entire contents of which is hereby incorporated by reference.

In certain embodiments, one or more of the heterologous antigens is from the principal neutralizing loop of a retrovirus, such as HIV-1 or HIV-2. For example, the heterologous antigen can be from the V3 loop of gp120 protein from HIV-1. A neutralizing loop can be identified by neutralizing antibodies, i.e., antibodies that neutralize infectivity of the virus. The sequences can be from any HIV strain known to one of skill in the art without limitation. Preferably, the HIV strain is a circulating strain. Such circulating strains include, for example, MN (e.g., subtype B) or Thai-E (e.g., subtype E). V3 loops of various strains of HIV-1 comprise about 35 amino acids. The strains of HIV can be T-cell tropic or macrophage tropic. In certain embodiments, the sequences from the V3 loop include at least about 8 amino acids (e.g., a peptide sufficiently long to fit into a Class II MHC binding pocket) that includes a V3 loop apex. The V3 loop of MN strain of HIV has the sequence: CTRPNYNKRKIGPGRAFYTTKNIIGTIRQAHC (SEQ ID NO.:3). The V3 loop of Thai-E strain of HIV has the sequence: CTRPSNNTRT SITIGPGQVFYRTGDIIGDI RKAYC (SEQ ID NO.:5). The V3 loop apex is underlined. The sequence can be about 14 to about 26 amino acids long, but is not limited to this size.

In other embodiments, one or more of the heterologous antigens can be an antigen expressed by a cell during disease. For example, one or more of the heterologous antigens can be a cancer-specific antigen. For example, certain breast cancers express a mutant EGF (“epidermal growth factor”) receptor that results from a splice variant. This mutant form is immunologically distinct from the wild-type EGF and therefore constitutes an attractive target for immunotherapy. Other suitable cancer-specific antigens include those that are expressed on the cell surface and, therefore, can be target of a cytotoxic T-lymphocyte response. Any such antigen known to one of skill in the art without limitation can be used as one or more of the heterologous antigens. For example, the cancer-specific antigen can be a prostate cancer-specific antigen (e.g., PSA), a breast cancer-specific marker (e.g., BRCA-1 or HER2), a pancreatic cancer-specific marker (e.g., CA9-19), a melanoma marker (e.g., tyrosinase) or a cancer-specific mutant form of EGF.

Other examples of cancer-specific antigens that can be used in the methods and compositions of the present invention include, but are not limited to, antigens from a cancer such as follicular lymphomas; carcinomas with p53 mutations; hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer; leukemia, including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia); polycythemia vera; lymphomas (e.g., Hodgkin's disease and non-Hodgkin's disease); multiple myeloma; Waldenstrom's macroglobulinemia; heavy chain disease; sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wiln's tumor, cervical cancer, testicular tumor, lung carcinoma, small cell lung carcinoma, bladder carcinoma, and epithelial carcinoma; glioma; astrocytoma; medulloblastoma; craniopharyngioma; ependymoma; pinealoma; hemangioblastoma; acoustic neuroma; oligodendroglioma; menangioma; melanoma; neuroblastoma; and retinoblastoma.

Specific antigens that have been identified as expressed on the surfaces of certain cancers and cell lines derived from such cancers are presented in Tables 1 and 2, below. The monoclona antibodies identified in the tables can be used as controls in experiments assessing the immune response induced by a chimeric immunogen of the invention against one or more of the antigens presented in Table 1 or 2. The fall citations for the references identified in Tables 1 and 2 may be found in U.S. Pat. No. 6,716,422, which is hereby incorporated by reference in its entirety. Additional antigens may be derived from a human or non-human cancer or tumor cell line described in U.S. Pat. No. 6,218,166, which is hereby incorporated by reference in its entirety.

TABLE 1
MARKER ANTIGENS OF SOLID TUMORS AND
CORRESPONDING MONOCLONAL ANTIBODIES

| | Antigen Identity/ | Monoclonal | |
|---|---|---|---|
| Tumor Site | Characteristics | Antibodies | Reference |
| A: GYNECOLOGICAL | ‘CA 125’ > 200 kD | OC 125 | Kabawat et al., 1983; |
| | | | Szymendera, 1986 |
| GY | mucin GP | | |
| ovarian | 80 Kd GP | OC 133 | Masuko et al, Cancer Res., |
| | | | 1984 |
| ovarian | ‘SGA’ 360 Kd GP | OMI | de Krester et al., 1986 |
| ovarian | High Mᵣ mucin | Mo v1 | Miotti et al, Cancer Res., |
| | | | 1985 |
| ovarian | High Mᵣ | Mo v2 | Miotti et al, Cancer Res., |
| | mucin/glycolipid | | 1985 |
| ovarian | NS | 3C2 | Tsuji et al., Cancer Res., |
| | | | 1985 |
| ovarian | NS | 4C7 | Tsuji et al., Cancer Res., |
| | | | 1985 |
| ovarian | High Mᵣ mucin | ID₃ | Gangopadhyay et al., 1985 |
| ovarian | High Mᵣ mucin | DU-PAN-2 | Lan et al., 1985 |
| GY | 7700 Kd GP | F 36/22 | Croghan et al., 1984 |
| ovarian | ‘gp 68’ 48 Kd GP | 4F₇/7A₁₀ | Bhattacharya et al., 1984 |
| GY | 40, 42 kD GP | OV-TL3 | Poels et al., 1986 |
| GY | ‘TAG-72’ High Mᵣ | B72.3 | Thor et al., 1986 |
| | mucin | | |
| ovarian | 300-400 Kd GP | DF₃ | Kufe et al., 1984 |
| ovarian | 60 Kd GP | 2C₈/2F₇ | Bhattacharya et al., 1985 |
| GY | 105 Kd GP | MF 116 | Mattes et al., 1984 |
| ovarian | 38-40 kD GP | MOv18 | Miotti et al., 1987 |
| GY | ‘CEA’ 180 Kd GP | CEA 11-H5 | Wagener et al., 1984 |
| ovarian | CA 19-9 or GICA | CA 19-9 | Atkinson et al., 1982 |
| | | (1116NS 19-9) | |
| ovarian | ‘PLAP’ 67 Kd GP | H17-E2 | McDicken et al., 1985 |
| ovarian | 72 Kd | 791T/36 | Perkins et al., 1985 |
| ovarian | 69 Kd PLAP | NDOG₂ | Sunderland et al., 1984 |
| ovarian | unknown Mᵣ PLAP | H317 | Johnson et al., 1981 |
| ovarian | p185^{HER2} | 4D5, 3H4, 7C2, | Shepard et al., 1991 |
| | | 6E9, 2C4, 7F3, | |
| | | 2H11, 3E8, | |
| | | 5B8, 7D3, SB8 | |
| uterus ovary | HMFG-2 | HMFG2 | Epenetos et al., 1982 |
| GY | HMFG-2 | 3.14.A3 | Burchell et al., 1983 |
| B: BREAST | 330-450 Kd GP | DF3 | Hayes et al., 1985 |
| | NS | NCRC-11 | Ellis et al., 1984 |
| | 37 kD | 3C6F9 | Mandeville et al., 1987 |
| | NS | MBE6 | Teramoto et al., 1982 |
| | NS | CLNH5 | Glassy et al., 1983 |
| | 47 Kd GP | MAC 40/43 | Kjeldsen et al., 1986 |
| | High Mᵣ GP | EMA | Sloane et al., 1981 |
| | High Mᵣ GP | HMFG1 | Arklie et al., 1981 |
| | | HFMG2 | |
| | NS | 3.15.C3 | Arklie et al., 1981 |
| | NS | M3, M8, M24 | Foster et al., 1982 |
| | 1 (Ma) blood group | M18 | Foster et al., 1984 |
| | Ags | | |
| | NS | 67-D-11 | Rasmussen et al., 1982 |
| | oestrogen receptor | D547Sp, | Kinsel et al., 1989 |
| | | D75P3, H222 | |
| | EGF Receptor | Anti-EGF | Sainsbury et al., 1985 |
| | Laminin Receptor | LR-3 | Horan Hand et al., 1985 |
| | erb B-2 p185 | TA1 | Gusterson et al., 1988 |
| | NS | H59 | Hendler et al., 1981 |
| | 126 Kd GP | 10-3D-2 | Soule et al., 1983 |
| | NS | HmAB1,2 | Imam et al., 1984; Schlom |
| | | | et al., 1985 |
| | NS | MBR 1,2,3 | Menard et al., 1983 |
| | 95 Kd | 24.17.1 | Thompson et al., 1983 |
| | 100 Kd | 24.17.2 (3E1.2) | Croghan et al., 1983 |
| | NS | F36/22.M7/105 | Croghan et al., 1984 |
| | 24 Kd | C11, G3, H7 | Adams et al., 1983 |
| | 90 Kd GP | B6.2 | Colcher et al., 1981 |
| | CEA & 180 Kd GP | B1.1 | Colcher et al., 1983 |
| | colonic & pancreatic | Cam 17.1 | Imperial Cancer Research |
| | mucin similar to Ca | | Technology MAb listing |
| | 19-9 | | |
| | milk mucin core | SM3 | Imperial Cancer Research |
| | | | Technology Mab listing |
| | protein milk mucin | SM4 | Imperial Cancer Research |
| | core | | Technology Mab listing |
| | protein affinity- | C-Mul (566) | Imperial Cancer Research |
| | purified milk | | Technology Mab listing |
| | mucin p185_{HER2} | 4D5 3H4, 7C2, | Shepard et al., 1991 |
| | | 6E9, 2C4, 7F3, | |
| | | 2H11, 3E8, | |
| | | 5B8, 7D3, 5B8 | |
| | CA 125 > 200 Kd GP | OC 125 | Kabawat et al., 1985 |
| | High Mᵣ | MO v2 | Miotti et al., 1985 |
| | mucin/glycolipid | | |
| | High Mᵣ mucin | DU-PAN-2 | Lan et al., 1984 |
| | ‘gp48’ 48 Kd GP | 4F₇/7A₁₀ | Bhattacharya et al., 1984 |
| | 300-400 Kd GP | DF₃ | Kufe et al., 1984 |
| | ‘TAG-72’ high Mᵣ | B72.3 | Thor et al., 1986 |
| | mucin | | |
| | ‘CEA’ 180 Kd GP | cccccCEA 11 | Wagener et al., 1984 |
| | ‘PLAP’ 67 Kd GP | H17-E2 | McDicken et al., 1985 |
| | HMFG-2 > 400 Kd | 3.14.A3 | Burchell et al., 1983 |
| | GP | | |
| | NS | FO23C5 | Riva et al., 1988 |
| C: COLORECTAL | TAG-72 High Mᵣ | B72.3 | Colcher et al., 1987 |
| | mucin | | |
| | GP37 | (17-IA) 1083-17-IA | Paul et al., 1986 |
| | Surface GP | C017-1A | LoBuglio et al., 1988 |
| | CEA | ZCE-025 | Patt et al., 1988 |
| | CEA | AB2 | Griffin et al., 1988a |
| | cell surface AG | HT-29-15 | Cohn et al., 1987 |
| | secretory epithelium | 250-30.6 | Leydem et al., 1986 |
| | surface glycoprotein | 44X14 | Gallagher et al., 1986 |
| | NS | A7 | Takahashi et al., 1988 |
| | NS | GA73.3 | Munz et al., 1986 |
| | NS | 791T/36 | Farrans et al., 1982 |
| | cell membrane & | 28A32 | Smith et al., 1987 |
| | cytoplasmic Ag | | |
| | CEA & vindesine | 28.19.8 | Corvalen, 1987 |
| | gp72 | X MMCO-791 | Byers et al., 1987 |
| | high Mᵣ mucin | DU-PAN-2 | Lan et al., 1985 |
| | high Mᵣ mucin | ID₃ | Gangopadhyay et al., 1985 |
| | CEA 180 Kd GP | CEA 11-H5 | Wagener et al., 1984 |
| | 60 Kd GP | 2C₈/2F₇ | Bhattacharya et al., 1985 |
| | CA-19-9 (or GICA) | CA-19-9 | Atkinson et al., 1982 |
| | | (1116NS 19-9) | |
| | Lewis a | PR5C5 | Imperial Cancer Research |
| | | | Technology Mab Listing |
| | Lewis a | PR4D2 | Imperial Cancer Research |
| | | | Technology Mab Listing |
| | colonic mucus | PR4D1 | Imperial Cancer Research |
| | | | Technology Mab Listing |
| D: MELANOMA | p97ₐ | 4.1 | Woodbury et al., 1980 |
| | p97ₐ | 8.2 M₁₇ | Brown, et al., 1981a |
| | p97_{b} | 96.5 | Brown, et al., 1981a |
| | p97_{c} | 118.1, 133.2, | Brown, et al., 1981a |
| | | (113.2) | |
| | p97_{c} | L₁, L₁₀, R₁₀ | Brown et al., 1981b |
| | | (R₁₉) | |
| | p97_{d} | I₁₂ | Brown et al., 1981b |
| | p97ₑ | K₅ | Brown et al., 1981b |
| | p155 | 6.1 | Loop et al., 1981 |
| | G_{D3} disialogan- | R24 | Dippold et al., 1980 |
| | glioside | | |
| | p210, p60, p250 | 5.1 | Loop et al., 1981 |
| | p280 p440 | 225.28S | Wilson et al., 1981 |
| | GP 94, 75, 70 & 25 | 465.12S | Wilson et al., 1981 |
| | P240-P250, P450 | 9.2.27 | Reisfeld et al., 1982 |
| | 100, 77, 75 Kd | F11 | Chee et al., 1982 |
| | 94 Kd | 376.96S | Imai et al., 1982 |
| | 4 GP chains | 465.12S | Imai et al., 1982; Wilson et |
| | | | al., 1981 |
| | GP 74 | 15.75 | Johnson & Reithmuller, |
| | | | 1982 |
| | GP 49 | 15.95 | Johnson & Reithmuller, |
| | | | 1982 |
| | 230 Kd | Me1-14 | Carrel et al., 1982 |
| | 92 Kd | Me1-12 | Carrel et al., 1982 |
| | 70 Kd | Me3-TB7 | Carrel et al., 1: 387, 1982 |
| | HMW MAA similar | 225.28SD | Kantor et al., 1982 |
| | to 9.2.27 AG | | |
| | HMW MAA similar | 763.24TS | Kantor et al., 1982 |
| | to 9.2.27 AG | | |
| | GP95 similar to | 705F6 | Stuhlmiller et al., 1982 |
| | 376.96S 465.12S | | |
| | GP125 | 436910 | Saxton et al., 1982 |
| | CD41 | M148 | Imperial Cancer Research |
| | | | Technology Mab listing |
| E: GASTROINTESINAL | high Mᵣ mucin | ID3 | Gangopadhyay et al., 1985 |
| pancreas, stomach | | | |
| gall bladder, pancreas, | high Mᵣ mucin | DU-PAN-2 | Lan et al., 1985 |
| stomach | | | |
| pancreas | NS | OV-TL3 | Poels et al., 1984 |
| pancreas, stomach, | ‘TAG-72’ high Mᵣ | B72.3 | Thor et al., 1986 |
| oesophagus | mucin | | |
| stomach | ‘CEA’ 180 Kd GP | CEA 11-H5 | Wagener et al., 1984 |
| pancreas | HMFG-2 > 400 Kd | 3.14.A3 | Burchell et al., 1983 |
| | GP | | |
| G.I. | NS | C COLI | Lemkin et al., 1984 |
| pancreas, stomach | CA 19-9 (Or GICA) | CA-19-9 | Szymendera, 1986 |
| | CA50 | (1116NS 19-9) | |
| | | and | |
| pancreas | CA125 GP | OC125 | Szymendera, 1986 |
| F: LUNG | p185^{HER2} | 4D5 3H4, 7C2, | Shepard et al., 1991 |
| | | 6E9, 2C4, 7F3, | |
| | | 2H1 1, 3E8, | |
| | | 5B8, 7D3, SB8 | |
| non-small cell lung | | | |
| carcinoma | | | |
| | high Mᵣ | MO v2 | Miotti et al., 1985 |
| | mucin/glycolipid | | |
| | ‘TAG-72’ high Mᵣ | B72.3 | Thor et al., 1986 |
| | mucin | | |
| | high Mr mucin | DU-PAN-2 | Lan et al., 1985 |
| | ‘CEA’ 180 kD GP | CEA 11-H5 | Wagener et al., 1984 |
| Malignant Gliomas | cytoplasmic antigen | MUC 8-22 | Stavrou, 1990 |
| | from 85HG-22 cells | | |
| | cell surface Ag from | MUC 2-3 | Stavrou, 1990 |
| | 85HG-63 cells | | |
| | cell surface Ag from | MUC 2-39 | Stavrou, 1990 |
| | 86HG-39 cells | | |
| | cell surface Ag from | MUC 7-39 | Stavrou, 1990 |
| | 86HG-39 cells | | |
| G: MISCELLANEOUS | p53 | PAb 240 | Imperial Cancer Research |
| | | | Technology MaB Listing |
| | | PAb 246 | |
| | | PAb 1801 | |
| small round cell | neural cell adhesion | ERIC.1 | Imperial Cancer Research |
| tumors | molecule | | Technology MaB Listing |
| medulloblastoma | | M148 | Imperial Cancer Research |
| neuroblastoma | | | Technology MaB Listing |
| | rhabdomyosarcoma | FMH25 | Imperial Cancer Research |
| | neuroblastoma | | Technology MaB Listing |
| renal cancer & | p155 | 6.1 | Loop et al., 1981 |
| glioblastomas | | | |
| bladder & laryngeal | “Ca Antigen” 350-390 | CA1 | Ashall et al., 1982 |
| cancers | kD | | |
| neuroblastoma | GD2 | 3F8 | Cheung et al., 1986 |
| Prostate | gp48 48 kD GP | 4F₇/7A₁₀ | Bhattacharya et al., 1984 |
| Prostate | 60 kD GP | 2C₈/2F₇ | Bhattacharya et al., 1985 |
| Thyroid | ‘CEA’ 180 kD GP | CEA 11-H5 | Wagener et al., 1984 |

TABLE 2
HUMAN TUMOR CELL LINES AND SOURCES

| ATTC HTB | | |
|---|---|---|
| NUMBER | CELL LINE | TUMOR TYPE |
| 1 | J82 | Transitional-cell carcinoma, bladder |
| 2 | RT4 | Transitional-cell papilloma, bladder |
| 3 | ScaBER | Squamous carcinoma, bladder |
| 4 | T24 | Transitional-cell carcinoma, bladder |
| 5 | TCCSUP | Transitional-cell carcinoma, bladder, primary grade IV |
| 9 | 5637 | Carcinoma, bladder, primary |
| 10 | SK-N-MC | Neuroblastoma, metastasis to supra-orbital area |
| 11 | SK-N-SH | Neuroblastoma, metastasis to bone marrow |
| 12 | SW 1088 | Astrocytoma |
| 13 | SW 1783 | Astrocytoma |
| 14 | U-87 MG | Glioblastoma, astrocytoma, grade III |
| 15 | U-118 MG | Glioblastoma |
| 16 | U-138 MG | Glioblastoma |
| 17 | U-373 MG | Glioblastoma, astrocytoma, grade III |
| 18 | Y79 | Retinoblastoma |
| 19 | BT-20 | Carcinoma, breast |
| 20 | BT-474 | Ductal carcinoma, breast |
| 22 | MCF7 | Breast adenocarcinoma, pleural effusion |
| 23 | MDA-MB-134-VI | Breast, ductal carcinoma, pleural effusion |
| 24 | MDA-MD-157 | Breast medulla, carcinoma, pleural effusion |
| 25 | MDA-MB-175-VII | Breast, ductal carcinoma, pleural effusion |
| 27 | MDA-MB-361 | Adenocarcinoma, breast, metastasis to brain |
| 30 | SK-BR-3 | Adenocarcinoma, breast, malignant pleural effusion |
| 31 | C-33 A | Carcinoma, cervix |
| 32 | HT-3 | Carcinoma, cervix, metastasis to lymph node |
| 33 | ME-180 | Epidermoid carcinoma, cervix, metastasis to omentum |
| 34 | MS751 | Epidermoid carcinoma, cervix, metastasis to lymph node |
| 35 | SiHa | Squamous carcinoma, cervix |
| 36 | JEG-3 | Choriocarcinoma |
| 37 | Caco-2 | Adenocarcinoma, colon |
| 38 | HT-29 | Adenocarcinoma, colon, moderately well-differentiated grade II |
| 39 | SK-CO-1 | Adenocarcinoma, colon, ascites |
| 40 | HuTu 80 | Adenocarcinoma, duodenum |
| 41 | A-253 | Epidermoid carcinoma, submaxillary gland |
| 43 | FaDu | Squamous cell carcinoma, pharynx |
| 44 | A-498 | Carcinoma, kidney |
| 45 | A-704 | Adenocarcinoma, kidney |
| 46 | Caki-1 | Clear cell carcinoma, consistent with renal primary, metastasis to |
| | | skin |
| 47 | Caki-2 | Clear cell carcinoma, consistent with renal primary |
| 48 | SK-NEP-1 | Wilms' tumor, pleural effusion |
| 49 | SW 839 | Adenocarcinoma, kidney |
| 52 | SK-HEP-1 | Adenocarcinoma, liver, ascites |
| 53 | A-427 | Carcinoma, lung |
| 54 | Calu-1 | Epidermoid carcinoma grade III, lung, metastasis to pleura |
| 55 | Calu-3 | Adenocarcinoma, lung, pleural effusion |
| 56 | Calu-6 | Anaplastic carcinoma, probably lung |
| 57 | SK-LU-1 | Adenocarcinoma, lung consistent with poorly differentiated, grade |
| | | III |
| 58 | SK-MES-1 | Squamous carcinoma, lung, pleural effusion |
| 59 | SW 900 | Squamous cell carcinoma, lung |
| 60 | EB1 | Burkitt lymphoma, upper maxilla |
| 61 | EB2 | Burkitt lymphoma, ovary |
| 62 | P3HR-1 | Burkitt lymphoma, ascites |
| 63 | HT-144 | Malignant melanoma, metastasis to subcutaueous tissue |
| 64 | Malme-3M | Malignnt melanoma, metastasis to lung |
| 66 | RPMI-7951 | Malignant melanoma, metastasis to lymph node |
| 67 | SK-MEL-1 | Malignant melanoma, metastasis to lymphatic system |
| 68 | SK-MEL-2 | Malignant melanoma, metastasis to skin of thigh |
| 69 | SK-MEL-3 | Malignant melanoma, metastasis to lymph node |
| 70 | SK-MEL-5 | Malignant melanoma, metastasis to axillary node |
| 71 | SK-MEL-24 | Malignant melanoma, metastasis to node |
| 72 | SK-MEL-28 | Malignant melanoma |
| 73 | SK-MEL-31 | Malignant melanoma |
| 75 | Caov-3 | Adenocarcinoma, ovary, consistent with primary |
| 76 | Caov-4 | Adenocarcinoma, ovary, metastasis to subserosa of fallopian tube |
| 77 | SK-OV-3 | Adenocarcinoma, ovary, malignant ascites |
| 78 | SW 626 | Adenocarcinoma, ovary |
| 79 | Capan-1 | Adenocarcinoma, pancreas, metastasis to liver |
| 80 | Capan-2 | Adenocarcinoma, pancrease |
| 81 | DU 145 | Carcinoma, prostate, metastasis to brain |
| 82 | A-204 | Rhabdomyosarcoma |
| 85 | Saos-2 | Osteogenic sarcoma, primary |
| 86 | SK-ES-1 | Anaplastic osteosarcoma versus Ewing sarcoma, bone |
| 88 | SK-LMS-1 | Leiomyosarcoma, vulva, primary |
| 91 | SW 684 | Fibrosarcoma |
| 92 | SW 872 | Liposarcoma |
| 93 | SW 982 | Axilla synovial sarcoma |
| 94 | SW 1353 | Chondrosarcoma, humerus |
| 96 | U-2 OS | Osteogenic sarcoma, bone primary |
| 102 | Malme-3 | Skin fibroblast |
| 103 | KATO III | Gastric carcinoma |
| 104 | Cate-1B | Embryonal carcinoma, testis, metastasis to lymph node |
| 105 | Tera-1 | Embryonal carcinoma, malignancy consistent with metastasis to |
| | | lung |
| 106 | Tera-2 | Embryonal carcinoma, malignancy consistent with, metastasis to |
| | | lung |
| 107 | SW579 | Thyroid carcinoma |
| 111 | AN3 CA | Endometrial adenocarcinoma, metastatic |
| 112 | HEC-1-A | Endometrial adenocarcinoma |
| 113 | HEC-1-B | Endometrial adenocarcinoma |
| 114 | SK-UT-1 | Uterine, mixed mesodermal tumor, consistent with |
| | | leiomyosarcoma grade III |
| 115 | SK-UT-1B | Uterine, mixed mesodermal tumor, consistent with |
| | | leiomyosarcoma grade III |
| 117 | SW 954 | Squamous cell carcinoma, vulva |
| 118 | SW 962 | Carcinoma, vulva, lymph node metastasis |
| 119 | NCI-H69 | Small cell carcinoma, lung |
| 120 | NCI-H128 | Small cell carcinoma, lung |
| 121 | BT-483 | Ductal carcinoma, breast |
| 122 | BT-549 | Ductal carcinoma, breast |
| 123 | DU4475 | Metastatic cutaneous nodule, breast carcinoma |
| 124 | HBL-100 | Breast |
| 125 | Hs 578Bst | Breast, normal |
| 126 | Hs 578T | Ductal carcinoma, breast |
| 127 | MDA-MB-330 | Carcinoma, breast |
| 128 | MDA-MB-415 | Adenocarcinoma, breast |
| 129 | MDA-MB-435S | Ductal carcinoma, breast |
| 130 | MDA-MB-436 | Adenocarcinoma, breast |
| 131 | MDA-MB-453 | Carcinoma, breast |
| 132 | MDA-MB-468 | Adenocarcinoma, breast |
| 133 | T-47D | Ductal carcinoma, breast, pleural effusion |
| 134 | Hs 766T | Carcinoma, pancreas, metastatic to lymph node |
| 135 | Hs 746T | Carcinoma, stomach, metastatic to left leg |
| 137 | Hs 695T | Amelanotic melanoma, metastatic to lymph node |
| 138 | Hs 683 | Glioma |
| 140 | Hs 294T | Melanoma, metastatic to lymph node |
| 142 | Hs 602 | Lymphoma, cervical |
| 144 | JAR | Choriocarcinoma, placenta |
| 146 | Hs 445 | Lymphoid, Hodgkin's disease |
| 147 | Hs 700T | Adenocarcinoma, metastatic to pelvis |
| 148 | H4 | Neuroglioma, brain |
| 151 | Hs 696 | Adenocarcinoma primary, unknown, metastatic to bone-sacrum |
| 152 | Hs 913T | Fibrosarcoma, metastatic to lung |
| 153 | Hs 729 | Rhabdomyosarcoma, left leg |
| 157 | FHs 738Lu | Lung, normal fetus |
| 158 | FHs 173We | Whole embryo, normal |
| 160 | FHs 738B1 | Bladder, normal fetus |
| 161 | NIH:0VCAR-3 | Ovary, adenocarcinoma |
| 163 | Hs 67 | Thymus, normal |
| 166 | RD-ES | Ewing's sarcoma |
| 168 | ChaGo | K-1 Bronchogenic carcinoma, subcutaneous metastasis, human |
| 169 | WERI-Rb-1 | Retinoblastoma |
| 171 | NCI-H446 | Small cell carcinoma, lung |
| 172 | NCI-H209 | Small cell carcinoma, lung |
| 173 | NCI-H146 | Small cell carcinoma, lung |
| 174 | NCI-H441 | Papillary adenocarcinoma, lung |
| 175 | NCI-H82 | Small cell carcinoma, lung |
| 176 | H9 | T-cell lymphoma |
| 177 | NCI-H460 | Large cell carcinoma, lung |
| 178 | NCI-H596 | Adenosquamous carcinoma, lung |
| 179 | NCI-H676B | Adenocarcinoma, lung |
| 180 | NCI-H345 | Small cell carcinoma, lung |
| 181 | NCI-H820 | Papillary adenocarcinoma, lung |
| 182 | NCI-H520 | Squamous cell carcinoma, lung |
| 183 | NCI-H661 | Large cell carcinoma, lung |
| 184 | NCI-H510A | Small cell carcinoma, extra-pulmonary origin, metastatic |
| 185 | D283 Med | Medulloblastoma |
| 186 | Daoy | Medulloblastoma |
| 187 | D341 Med | Medulloblastoma |
| 188 | AML-193 | Acute monocyte leukemia |
| 189 | MV4-11 | Leukemia biphenotype |

Further, in certain embodiments, one or more of the heterologous antigens can be a molecule that potentiates an immune response against another heterologous antigen. Any antigen that can act as immune stimulant known by one of skill in the art without limitation can be used as an antigen in such embodiments. For example, the heterologous antigen can be a nucleic acid with an unmethylated CpG motif, with a methylated CpG motif, or without any CpG motifs, as described in U.S. Pat. Nos. 6,653,292 and 6,239,116 and Published U.S. Application 20040152649, lipopolysaccharide (LPS) or an LPS derivative such as mono- or diphosphoryl lipid A, or any of the LPS derivatives or other adjuvants described in U.S. Pat. Nos. 6,716,623, 6,720,146, and 6,759,241.

Still further, in certain embodiments, one or more of the heterologous antigens can be a well-characterized test antigen. For example, in certain embodiments, one or more heterologous antigen can be ovalbumin, or a portion thereof. In certain embodiments, one or more heterologous antigen can be hen egg-white lysozyme. Any well-characterized test antigen without limitation can be used in the chimeric immunogen in such embodiments.

5.2.4. Endoplasmic Reticulum Retention Domain

The chimeric immunogens of the invention can optionally comprise an endoplasmic reticulum retention domain. This domain comprises an endoplasmic reticulum signal sequence, which functions in trafficking the chimeric immunogen from the endosome to the endoplasmic reticulum, and from thence into the cytosol. Native PE comprises an ER retention domain in domain III. The ER retention domain comprises an ER retention signal sequence at its carboxy terminus. In native PE, this ER retention signal is REDLK (SEQ ID NO.:6). The terminal lysine can be eliminated (i.e., REDL (SEQ ID NO.:7)) without an appreciable decrease in activity. However, any ER retention signal sequence known to one of skill in the art without limitation can be used in the chimeric immunogens of the invention. Other suitable ER retention signal sequences include, but are not limited to, KDEL (SEQ ID NO.:8), or dimers or multimers of these sequences. See Ogata et al., 1990, J. Biol. Chem. 265:20678-85; U.S. Pat. No. 5,458,878; and Pastan et al., 1992, Annu. Rev. Biochem. 61:331-54.

In certain embodiments, the chimeric immunogen comprises domain III of native PE, or a portion thereof. Preferably, the chimeric immunogen comprises domain III of ΔE553 PE. In certain embodiments, domain III, including the ER retention signal, can be entirely eliminated from the chimeric immunogen. In other embodiments, the chimeric immunogen comprises an ER retention signal sequence and comprises a portion or none of the remainder of PE domain III. In certain embodiments, the portion of PE domain III other than the ER retention signal can be replaced by another amino acid sequence. This amino acid sequence can itself be non immunogenic, slightly immunogenic, or highly immunogenic. A highly immunogenic ER retention domain is preferable for use in eliciting a humoral immune response. For example, PE domain III is itself highly immunogenic and can be used in chimeric immunogens where a robust humoral immune response is desired. Chimeras in which the ER retention domain is only slightly immunogenic are preferred when a Class I MHC-dependent cell-mediated immune response is desired.

ER retention domain activity can routinely be assessed by those of skill in the art by testing for translocation of the protein into the target cell cytosol using, for example, the assays described below.

In native PE, the ER retention sequence is located at the C-terminus of domain III. Native PE domain III has at least two observable activities. Domain III mediates ADP-ribosylation and therefore toxicity. Further, the ER retention signal present at the C-terminus directs endocytosed toxin into the endoplasmic reticulum and from thence, into the cytosol. Eliminating the ER retention sequence from the chimeric immunogens does not alter the activity of Pseudomonas exotoxin as a superantigen, but does prevent it from eliciting an MHC Class I-dependent cell-mediated immune response.

The PE domain that mediates ADP-ribosylation is located between about amino acids 400 and 600 of PE. This toxic activity of native PE is preferably eliminated in the chimeric immunogens of the invention. By doing so, the chimeric immunogen can be used as a vehicle for delivering heterologous antigens to be processed by the cell and presented on the cell surface with MHC Class I or Class II molecules, as desired, rather than as a toxin. ADP ribosylation activity can be eliminated by, for example, deleting amino acid E553. See, e.g., Lukac et al., 1988, Infect. Immun. 56:3095-3098. Alternatively, the amino acid sequence of domain III, or portions of it, can be deleted from the protein. Of course, an ER retention sequence should be included at the C-terminus if a Class I MHC-mediated immune response is to be induced.

In certain embodiments, the ER retention domain is the native amino acid sequences of PE domain III, or a fragment thereof, with one, two, three, four, five, eight, ten, fifteen, twenty, thirty, forty, fifty, or more conservative or nonconservative substiutions, additions, or deletions. In certain embodiments, the ER retention domain is domain III of PE. In other embodiments, the ER retention domain is domain III of ΔE553 PE. In still other embodiments, the ER retention domain comprises an amino acid sequence that is selected from the group consisting of RDELK, RDEL, and KDEL.

### 5.3. Methods for Inducing an Immune Response

In another aspect, the invention provides methods of inducing an immune response. The methods allow one of skill in the art to induce a cellular, humoral, and/or secretory immune response. Further, by including antigens that can induce one or more of these immune responses in a chimeric immunogen, the immune response that is induced can be a cellular and humoral, secretory and humoral, cellular and secretory, or cellular, humoral, and secreorty immune response. These methods generally rely on administration of a chimeric immunogen of the invention to a subject in whom the immune response is to be induced. As described above, the chimeric immunogens can be used to induce an immune response that is specific for at least one of two or more heterologous antigens present in the chimeric immunogen. In certain embodiments, the immune response that is induced is a prophylactic immune response, i.e., the subject is not already afflicted with a disease caused by an agent from which at least one of the heterologous antigens is derived. In other embodiments, the immune response that is induced is therapeutic, i.e., the subject is already afflicted with a disease caused by an agent from which at least one of the heterologous antigens is derived.

Accordingly, the invention provides methods for inducing an immune response against at least one of two or more heterologous antigens. In certain embodiments, the methods comprise administering to a subject in whom the immune response is to be induced a chimeric immunogen bearing the two or more heterologous antigens. The chimeric immunogen can be administered as a composition, as described below. In the case of an infection by a pathogen, the resultant immune responses protect against infection by a pathogen bearing at least one of the heterologous antigens or against cells that express at least one of the heterologous antigens. For example, if the pathology results from bacterial or parasitic protozoan infection, the immune response is mounted against the pathogens, themselves. If the pathogen is a virus, infected cells will express at least one of the heterologous antigens on their surface and become the target of a cell mediated immune response, though there can also be an immune response mounted against viral particles. Aberrant cells, such as cancer cells, that express antigens not present on the surface of normal cells also can be subject to a cell mediated immune response and/or humoral immune response.

5.3.1. Humoral Immune Responses

In certain embodiments, the invention provides a method for inducing a humoral immune response in a subject against at least one of two or more heterologous antigens. The methods generally comprise administering to a subject a chimeric immunogen that is configured to produce a humoral immune response. Such immune responses generally involve the production of antibodies specific for the antigen or antigens. Certain embodiments of the chimeric immunogens have properties that allow the skilled artisan to induce a humoral immune response against at least one of the heterologous antigens. For example, when one or more of the heterologous antigens is inserted into PE domain Ib, the flanking cysteines cause the heterologous antigen(s) to be extended from the remainder of the immunogen and facilitate recognition of the antigen(s) by a B cell through an interaction with a B-cell receptor. Interaction between the heterologous antigen(s) and the B cell receptor stimulates clonal expansion of the B cell bearing the receptor, eventually resulting in a population of plasma cells that secrete antibodies specific for the antigen(s).

In most circumstances, B cell recognition of antigen is necessary, but not sufficient, to induce a robust humoral immune response. The humoral response is greatly potentiated by CD4⁺ (helper) T cell signaling to B cells primed by antigen recognition. Helper T cells are activated to provide such signals to B cells by recognition of antigen processed through the Class II MHC pathway. The heterologous antigen recognized by the T cell can, but need not, be the same heterologous antigen that is recognized by the B cell. The chimeric immunogens of the invention can be targeted to such antigen presenting cells for processing in the Class II MHC pathway in order to stimulate helper T cells to activate B cells. By doing so, the chimeric immunogens can be used to stimulate a robust humoral immune response that is specific for at least one of the heterologous antigens.

The chimeric immunogens can all be utilized for inducing a humoral immune response against one or more heterologous antigens that are constrained within their native environment. By inserting one or more of such heterologous antigens into the Ib loop of PE-based chimeric immunogens, the antigen(s) can be presented to immune cells in native or near-native conformation. The resulting antibodies generally recognize the native antigen(s) better than those raised against unconstrained versions of the heterologous antigen(s). The Ib loop can also be used to present one or more B cell antigens that are not constrained in their native environment. In such embodiments, the antigen(s) inserted into the Ib loop should be flanked by a sufficient number of, e.g., about three to about five, about three to about eight, about five to about ten, amino acids that give conformational flexibility, such as, e.g., glycine, serine, etc., to allow the antigen(s) to fold into its native form and avoid constraint by the disulfide linkage between the cysteines of the Ib loop.

The humoral immune response induced by the chimeric immunogens can be assessed using any method known by one of skill in the art without limitation. For example, an animal's immune response against one or more of the heterologous antigens can be monitored by taking test bleeds and determining the titer of antibody reactivity to the heterologous antigen(s). When appropriately high titers of antibody to the heterologous antigen(s) are obtained, blood can be collected from the animal and antisera prepared. The antisera can be further enriched for antibodies reactive to the heterologous antigen(s), when desired. See, e.g., Coligan, 1991, Current Protocols in Immunology, Greene Publishing Associates and Wiley Interscience, NY; and Harlow and Lane, 1989, Antibodies: A Laboratory Manual, Cold Spring Harbor Press, NY.

Antibodies produced in response to administration of the chimeric immunogens can then be used for any purpose known by one of skill in the art, without limitation. For example, the antibodies can be used to make monoclonal antibodies, humanized antibodies, chimeric antibodies or antibody fragments. Techniques for producing such antibody derivatives may be found in, for example, Stites et al. eds., 1997, Medical Immunology (9th ed.), McGraw-Hill/Appleton & Lange, CA; Harlow and Lane, 1989, Antibodies: A Laboratory Manual, Cold Spring Harbor Press, NY; Goding, 1986, Monoclonal Antibodies: Principles and Practice (2d ed.), Academic Press, NY; Kohler and Milstein, 1975, Nature 256: 495-497; and U.S. Pat. No. 5,585,089.

5.3.2. Cell-Mediated Immune Responses

In other embodiments, the invention provides methods for eliciting a cell-mediated immune response against cells expressing at least one of two or more heterologous antigens. The methods generally comprise administering to a subject a chimeric immunogen that comprises two or more heterologous antigens that is configured to produce a cell-mediated immune response. Such immune responses generally involve the activation of cytotoxic T lymphocytes that can recognize and kill cells that display the antigen on their surfaces. However, certain aspects of humoral immune responses give rise to cell-mediated effects as well, as described below. Certain embodiments of the chimeric immunogens have properties that allow the skilled artisan to induce a cell-mediated immune response against at least one of the heterologous antigens.

In particular, heterologous antigens that are inserted into a chimeric immunogen near, e.g., between about one to about 20, about 10 to about 50, or about one to about 100 amino acids from, an ER retention signal tend to induce a cell-mediated immune response. Without intending to be bound to any particular theory or mechanism of action, it is believed that the ER retention signal causes the chimeric immunogen to be trafficked from an endosome to the ER, and from thence into the cytosol. Once in the cytosol, peptides from the immunogen, including the heterologous antigen, enter the Class I MHC processing pathway. The peptides associate with Class I MHC and are presented on the surface of the cell into which the immunogen has been introduced. CD8⁺ (cytotoxic) T lymphocytes then recognize the heterologous antigen in association with Class I MHC and thereby become activated and primed to kill cells that similarly have the heterologous antigen associated with Class I MHC on their surfaces.

Without intending to be bound to any particular theory or mechanism of action, art of the processing that occurs during presentation on Class I MHC is believed to result in degradation of the chimeric immunogen into peptides that can associate with the MHC molecule. This proteolysis begins in the endosome and continues in the cytosol. If, in the course of this process, the heterologous antigen is separated from the ER retention signal before the heterologous antigen is trafficked to the cytosol, it is believed that the heterologous antigen cannot associate with Class I MHC. In such circumstances, the heterologous antigen remains in the endosome, and is directed to the Class II MHC processing pathway. Accordingly, it is believed that the distance, e.g., the number of amino acids, between the heterologous antigen and the ER retention signal can affect the degree to which the antigen is presented in association with Class I or Class II MHC.

Thus, the skilled artisan can place the heterologous antigens in the chimeric immunogen according to this guidance to induce the immune response that is intended. For example, where the chimeric immunogen comprises two heterologous antigens, each of which is intended to induce a cell-mediated immune response, both heterologous antigens can be placed near the ER retention signal. Conversely, when the chimeric immunogen comprises two heterologous antigens, one of which is intended to elicit a cell mediated immune response, and one of which is not, the heterologous antigens should be oriented appropriately. The skilled artisan, using the assays described below, can routinely test such chimeric immunogen to assess the nature and specificity of the immune response elicited by the immunogen to ensure that the immune response is of the type desired to be induced.

Features of peptides that associate with the various allelic forms of Class I MHC have been well characterized. For example, peptides bound by HLA-A1 generally comprise a first conserved residue of T, S or M, a second conserved residue of D or E, and a third conserved residue of Y, wherein the first and second residues are adjacent, and both are separated from the third residue by six or seven amino acids. Peptides that bind to other alleles of Class I MHC have also been characterized. Using this knowledge, the skilled artisan can select heterologous antigens that can associate with a Class I MHC allele that is expressed in the subject. By administering chimeric immunogens comprising such antigens near the ER retention signal, a cell-mediated immune response can be induced against the antigens near the ER retention signal.

Further, much like humoral immune responses mediated by B-cells, cell-mediated immune responses mediated by cytotoxic T cells or other immune effector cells can be potentiated by activated helper T cells. Thus, one or more of the heterologous antigens in the chimeric immunogens configured to elicit a cell-mediated response can also comprise a Class II MHC antigen along with the Class I MHC antigen. Of course, the Class II MHC antigen should be oriented relative to the ER retention signal to allow processing and presentation of the Class II MHC antigen on Class II MHC.

Cell-mediated immune responses can also arise as a consequence of humoral immune responses. Antibodies produced in the course of the humoral immune response bind to their cognate antigen; if this antigen is present on the surface of a cell, the antibody binds to the cell surface. Cells bound by antibodies in this manner are subject to antibody-dependent cell-mediated cytotoxicity, in which immune cells that bear Fc receptors attack the marked cells. For example, natural killer cells and macrophages have Fc receptors and can participate in this phenomenon.

5.3.3. Secretory Immune Response

In other embodiments, the invention provides methods for eliciting a secretory immune response against at least one of two or more heterologous antigens. The methods generally comprise administering to a mucous membrane of the subject a chimeric immunogen that comprises two or more heterologous antigens, wherein the chimeric immunogen is configured to bind to a receptor present on the mucous membrane. The mucous membrane can be any mucous membrane known by one of skill in the art to be present in the subject, without limitation. For example, the mucous membrane can be present in the eye, nose, mouth, trachea; lungs, esophagus, stomach, small intestine, large intestine, rectum, anus, sweat glands, vulva, vagina, or penis of the subject. Certain embodiments of the chimeric immunogens have properties that allow the skilled artisan to induce a secretory immune response against at least one of the heterologous antigens.

In particular, chimeric immunogens that comprise receptor binding domains that can bind to a receptor present on the apical membrane of an epithelial cell can be used to induce a secretory immune response. Such receptor binding domains are extensively described above. Without intending to be bound by any particular theory or mechanism of action, it is believed that the original encounter with the antigen at the mucosal surface directs the immune system to produce a secretory rather than humoral immune response.

Secretory immune responses are desirable for protecting against any pathogen that enters the body through a mucous membrane. Mucous membranes are primary entryways for many infectious pathogens, including, for example, HIV, herpes, vaccinia, cytomegalovirus, yersinia, vibrio, and Pseudomonas spp. Mucous membranes can be found in the mouth, nose, throat, lung, vagina, rectum and colon. As one defense against entry by these pathogens, the body secretes secretory IgA from mucosal epithelial membranes that can bind the pathogens and prevent or deter pathogenesis. Furthermore, antigens presented at one mucosal surface can trigger responses at other mucosal surfaces due to trafficking of antibody-secreting cells between the mucous membranes. The structure of secretory IgA appears to be crucial for its sustained residence and effective function at the luminal surface of a mucous membrane. “Secretory IgA” or “sIgA” generally refers to a polymeric molecule comprising two IgA immunoglobulins joined by a J chain and further bound to a secretory component. While mucosal administration of antigens can generate an IgG response, parenteral administration of immunogens rarely produces strong sIgA responses.

The chimeric immunogens can be administered to the mucous membrane of the subject by any suitable method or in any suitable formulation known to one of skill in the art without limitation. For example, the chimeric immunogens can be administered in the form of liquids or solids, e.g., sprays, ointments, suppositories or erodible polymers impregnated with the immunogen. Administration can involve applying the immunogen to a one or more different mucosal surface(s). Further, in certain embodiments, the chimeric immunogen can be administered in a single dose. In other embodiments, the chimeric immunogen, can be administered in a series of two or more administrations. In certain embodiments, the second or subsequent administration of the chimeric immunogen is administered parenterally, e.g., subcutaneously or intramuscularly.

The sIgA response is strongest on mucosal surfaces exposed to the immunogen. Therefore, in certain embodiment, the immunogen is applied to a mucosal surface that is likely to be a site of exposure to the pathogen. Accordingly, chimeric immunogens against pathogens encountered on vaginal, anal, or oral mucous membranes are preferably administered to vaginal, anal or oral mucosal surfaces, respectively. However, nasal administration of the chimeric immunogens can also induce robust secretory immune responses from other mucous membranes. See, for example, Boyaka et al., 2003, Cur. Pharm. Des. 9:1965-1972.

Mucosal administration of the chimeric immunogens of this invention results in strong memory responses, both for IgA and IgG. These memory responses can advantageously be boosted by re-administering the chimeric immunogen after a period of time. Such booster administrations can be administered either mucosally or parenterally. The memory response can be elicited by administering a booster dose more than a year after the initial dose. For example, a booster dose can be administered about 12, about 16, about 20 or about 24 months after the initial dose.

### 5.4. Polynucleotides Encoding Chimeric Immunogens

In another aspect, the invention provides polynucleotides comprising a nucleotide sequence encoding a chimeric immunogen of the invention. These polynucleotides are useful, for example, for making the chimeric immunogens. In yet another aspect, the invention provides an expression system that comprises a recombinant polynucleotide sequence encoding a receptor binding domain, a translocation domain, an optional ER retention domain, and one, two, or more insertion site(s) for a polynucleotide sequence encoding a heterologous antigen. The insertion site(s) can be anywhere in the polynucleotide sequence so long as the insertion does not disrupt, e.g., completely ablate the activity of, the receptor binding domain, the translocation domain, or the optional ER retention domain. Preferably, one of the insertion sites is between the translocation domain and the ER retention domain. In other equally preferred embodiments, one of the insertion sites is in the ER retention domain.

In certain embodiments, the recombinant polynucleotides are based on polynucleotides encoding PE, or portions or derivatives thereof. In other embodiments, the recombinant polynucleotides are based on polynucleotides that hybridize to a polynucleotide that encodes PE under stringent hybridization conditions. A nucleotide sequence encoding PE is presented as SEQ ID NO.:9. This sequence can be used to prepare PCR primers for isolating a nucleic acid that encodes any portion of this sequence that is desired. For example, PCR can be used to isolate a nucleic acid that encodes one or more of the functional domains of PE. A nucleic acid so isolated can then be joined to nucleic acids encoding other functional domains of the chimeric immunogens using standard recombinant techniques.

Other in vitro methods that can be used to prepare a polynucleotide encoding PE, PE domains, or any other functional domain useful in the chimeric immunogens of the invention include, but are not limited to, reverse transcription, the polymerase chain reaction (PCR), the ligase chain reaction (LCR), the transcription-based amplification system (TAS), the self-sustained sequence replication system (3SR) and the QP replicase amplification system (QB). Any such technique known by one of skill in the art to be useful in construction of recombinant nucleic acids can be used. For example, a polynucleotide encoding the protein or a portion thereof can be isolated by polymerase chain reaction of cDNA using primers based on the DNA sequence of PE or another polynucleotide encoding a receptor binding domain.

Guidance for using these cloning and in vitro amplification methodologies are described in, for example, U.S. Pat. No. 4,683,195; Mullis et al., 1987, Cold Spring Harbor Symp. Quant. Biol. 51:263; and Erlich, ed., 1989, PCR Technology, Stockton Press, NY. Polynucleotides encoding a chimeric immunogen or a portion thereof also can be isolated by screening genomic or cDNA libraries with probes selected from the sequences of the desired polynucleotide under stringent, moderately stringent, or highly stringent hybridization conditions.

Construction of nucleic acids encoding the chimeric immunogens of the invention can be facilitated by introducing one, two, or more insertion site(s) for a nucleic acid encoding a heterologous antigen into the construct. In certain embodiments, an insertion site for a heterologous antigen can be introduced between the nucleotides encoding the cysteine residues of domain Ib. In other embodiments, an insertion site can be introduced anywhere in the nucleic acid encoding the immunogen so long as the insertion does not disrupt the functional domains encoded thereby. In certain embodiments, an insertion site can be in the ER retention domain. In certain embodiments, an insertion site is introduced into the nucleic acid encoding the chimeric immunogen. In other embodiments, a nucleic acid comprising an insertion site can replace a portion of the nucleic acid encoding the immunogen, as long as the replacement does not disrupt the receptor binding domain or the translocation domain.

In more specific embodiments, at least one of the insertion sites comprises a cloning site cleaved by a restriction enzyme. In certain embodiments, the cloning site can be recognized and cleaved by a single restriction enzyme, for example, by PstI. In such examples, a polynucleotide encoding heterologous antigen that is flanked by PstI sequences can be inserted into the vector. In other embodiments, at least one of the insertion sites comprises a polylinker that comprises one, two, three, four, five, ten, or more cloning sites, each of which can be cleaved by one or more restriction enzymes.

Further, the polynucleotides can also encode a secretory sequence at the amino terminus of the encoded chimeric immunogen. Such constructs are useful for producing the chimeric immunogens in mammalian cells as they simplify isolation of the immunogen.

Furthermore, the polynucleotides of the invention also encompass derivative versions of polynucleotides encoding a chimeric immunogen. Such derivatives can be made by any method known by one of skill in the art without limitation. For example, derivatives can be made by site-specific mutagenesis, including substitution, insertion, or deletion of one, two, three, five, ten or more nucleotides, of polynucleotides encoding the chimeric immunogen. Alternatively, derivatives can be made by random mutagenesis. One method for randomly mutagenizing a nucleic acid comprises amplifying the nucleic acid in a PCR reaction in the presence of 0.1 mM MnCl₂ and unbalanced nucleotide concentrations. These conditions increase the misincorporation rate of the polymerase used in the PCR reaction and result in random mutagenesis of the amplified nucleic acid.

Several site-specific mutations and deletions in chimeric molecules derived from PE have been made and characterized. For example, deletion of nucleotides encoding amino acids 1-252 of PE yields a construct referred to as “PE40.” Deleting nucleotides encoding amino acids 1-279 of PE yields a construct referred to as “PE37.” See U.S. Pat. No. 5,602,095. In both of these constructs, the receptor binding domain of PE, i.e., domain Ia, has been deleted. Nucleic acids encoding a receptor binding domain can be ligated to these constructs to produce chimeric immunogens that are targeted to the cell surface receptor recognized by the receptor binding domain. Of course, these constructs are particularly useful for expressing chimeric immunogens that have a receptor binding domain that is not domain Ia of PE. The constructs can optionally encode an amino-terminal methionine to assist in expression of the construct. In certain embodiments, the receptor binding domain can be ligated to the 5′ end of the polynucleotide encoding the translocation domain and optional ER retention domain. In other embodiments, the polynucleotide can be inserted into the constructs in the nucleotide sequence encoding the ER retention domain.

Other nucleic acids encoding mutant forms of PE that can be used as a source of nucleic acids for constructing the chimeric immunogens of the invention include, but are not limited to, PEΔ553 and those described in U.S. Pat. Nos. 5,602,095; 5,512,658 and 5,458,878, and in Vasil et al., 1986, Infect. Immunol. 52:538-48.

### 5.5. Expression Vectors

In still another aspect, the invention provides expression vectors for expressing the chimeric immunogens. Generally, expression vectors are recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

Useful promoters for use in expression vectors include, but are not limited to, a metallothionein promoter, a constitutive adenovirus major late promoter, a dexamethasone-inducible MMTV promoter, a SV40 promoter, a MRP pol III promoter, a constitutive MPSV promoter, a tetracycline-inducible CMV promoter (such as the human immediate-early CMV promoter), and a constitutive CMV promoter.

The expression vectors should contain expression and replication signals compatible with the cell in which the chimeric immunogens are expressed. Expression vectors useful for expressing chimeric immunogens include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNA1 (Invitrogen, San Diego, Calif.), in which the expression control sequence comprises the CMV promoter, provides good rates of transfection and expression into such cells.

In certain embodiments, the expression vectors comprise one or more insertion sites that contain one, two, three, four, five, eight, ten, or more sequences recognized and cleaved by restriction enzymes to facilitate convenient insertion of a nucleic acid sequence encoding a peptide heterologous antigen. In certain embodiments, the insertion site is inserted into a portion of the expression vector that encodes Domain Ib of PE. In certain embodiments, the insertion site replaces all or a portion of a region of the expression vector that encodes Domain Ib of PE. In certain embodiments, the insertion site is inserted into a portion of the expression vector that encodes Domain III of PE. In certain embodiments, the insertion site replaces all or a portion of a region of the expression vector that encodes Domain III of PE. In certain embodiments, the insertion site is inserted into a portion of the expression vector that encodes Domain Ia of PE. In certain embodiments, the insertion site is inserted into a portion of the expression vector that encodes Domain II of PE. Of course, where the insertion site is inserted into a region of the expression vector that encodes a functional portion of the chimeric immunogen, introduction of the insertion site should be selected to avoid disrupting the activity of such functional regions.

The expression vectors can be introduced into the cell for expression of the chimeric immunogens by any method known to one of skill in the art without limitation. Such methods include, but are not limited to, e.g., direct uptake of the molecule by a cell from solution; facilitated uptake through lipofection using, e.g., liposomes or immunoliposomes; particle-mediated transfection; etc. See, e.g., U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY.

The expression vectors can also contain a purification moiety that simplifies isolation of the protein. For example, a polyhistidine moiety of, e.g., six histidine residues, can be incorporated at the amino terminal end of the protein. The polyhistidine moiety allows convenient isolation of the protein in a single step by nickel-chelate chromatography. In certain embodiments, the purification moiety can be cleaved from the remainder of the chimeric immunogen following purification. In other embodiments, the moiety does not interfere with the function of the functional domains of the chimeric immunogen and thus need not be cleaved.

### 5.6. Cell for Expressing a Chimeric Immunogen

In yet another aspect, the invention provides a cell comprising an expression vector for expression of the chimeric immunogens, or portions thereof. The cell is preferably selected for its ability to express high concentrations of the chimeric immunogen to facilitate purification of the protein. In certain embodiments, the cell is a prokaryotic cell, for example, E. coli. As described in the examples, the chimeric immunogens are properly folded and comprise the appropriate disulfide linkages when expressed in E. coli.

In other embodiments, the cell is a eukaryotic cell. Useful eukaryotic cells include yeast and mammalian cells. Any mammalian cell known by one of skill in the art to be useful for expressing a recombinant polypeptide, without limitation, can be used to express the chimeric immunogens. For example, Chinese hamster ovary (CHO) cells can be used to express the chimeric immunogens.

### 5.7. Compositions Comprising Chimeric Immunogens and Uses Thereof

In yet another aspect, the invention provides compositions comprising one or more chimeric immunogens. The compositions are useful for eliciting a protective immune response against at least one of the heterologous antigens, particularly against pathogens or cells bearing at least one of the heterologous antigens. A composition can include one or a plurality of chimeric immunogens. For example, a composition can include chimeric immunogens with two or more heterologous antigens from several circulating strains of a pathogen. As the pathogen changes, additional chimeric immunogens can be constructed that include the altered antigens, for example, from breakthrough viruses.

5.7.1. Compositions Comprising Chimeric Immunogens

The chimeric immunogens of the invention can be formulated in compositions. The compositions are generally formulated appropriately for the immediate use intended for the composition. For example, if the chimeric immunogen is not to be administered immediately, the chimeric immunogen can be formulated in a composition suitable for storage. One such composition is a lyophilized preparation of the chimeric immunogen together with a suitable stabilizer. Alternatively, the chimeric immunogen can be formulated as a composition for storage in a solution with one or more suitable stabilizers. Any such stabilizer known to one of skill in the art without limitation can be used. For example, stabilizers suitable for lyophilized preparations include, but are not limited to, sugars, salts, surfactants, proteins, chaotropic agents, lipids, and amino acids. Stabilizers suitable for liquid preparations include, but are not limited to, sugars, salts, surfactants, proteins, chaotropic agents, lipids, and amino acids. Specific stabilizers than can be used in the compositions include, but are not limited to, trehalose, serum albumin, phosphatidylcholine, lecithin, and arginine. Other compounds, compositions, and methods for stabilizing a lyophilized or liquid preparation of the delivery constructs may be found, for example, in U.S. Pat. Nos. 6,573,237, 6,525,102, 6,391,296, 6,255,284, 6,133,229, 6,007,791, 5,997,856, and 5,917,021.

Further, the compositions of the invention can be formulated for administration to a subject. The formulation can be suitable for administration to a nasal, oral, vaginal, rectal, or other mucosal surface. Such compositions generally comprise one or more chimeric immunogens of the invention and a pharmaceutically acceptable excipient, diluent, carrier, or vehicle. Any such pharmaceutically acceptable excipient, diluent, carrier, or vehicle known to one of skill in the art without limitation can be used. Examples of a suitable excipient, diluent, carrier, or vehicle can be found in Remington's Pharmaceutical Sciences, 20th Ed. 2000, Mack Publishing Co., Easton.

In certain embodiments, the compositions comprise about 1, about 5, about 10, about 20, about 30, about 40, or about 50 mM sodium chloride. Pseudomonas appears to bind epithelial cells via the pilin-asialo-GM1 interaction more efficiently in environments comprising 100 mM NaCl. By reducing the salt concentration, the chimeric immunogen is believed to be more likely to bind to an epithelial cell through its receptor binding domain rather through a pilin-asialo-GM1 interaction. By increasing the proportion of chimeric immunogen bound via the receptor binding domain, a higher concentration of chimeric immunogen can be delivered to the bloodstream of the subject.

The compositions can also include an adjuvant that potentiates an immune response when used in administered in conjunction with the chimeric immunogen. Useful adjuvants, particularly for administration to human subjects, include, but are not limited to, alum, aluminum hydroxide or aluminum phosphate. Other suitable adjuvants are described in Sheikh et al., 2000, Cur. Opin. Mol. Ther. 2:37-54. Still other useful adjuvants include a nucleic acid with an unmethylated CpG motif, with a methylated CpG motif, or without any CpG motifs, as described in U.S. Pat. Nos. 6,653,292 and 6,239,116 and Published U.S. Application 20040152649; lipopolysaccharide (LPS) or an LPS derivative such as mono- or diphosphoryl lipid A; and any of the LPS derivatives or other adjuvants described in U.S. Pat. Nos. 6,716,623, 6,720,146, and 6,759,241. Adjuvants are most useful when the composition is to be injected rather than administered to a mucosal membrane of the subject. However, adjuvants are known that can potentiate an immune response when compositions that comprise the adjuvant are administered to a mucosal surface. See, e.g., U.S. Pat. Nos. 6,525,028, 6,544,518, and 6,649,172.

In certain embodiments, the compositions are formulated for oral administration. In such embodiments, the compositions are formulated to protect the chimeric immunogen from acid and/or enzymatic degradation in the stomach. Upon passage to the neutral to alkaline environment of the duodenum, the chimeric immunogen then contacts a mucous membrane and is transported across the polarized epithelial membrane. The delivery constructs may be formulated in such compositions by any method known by one of skill in the art, without limitation.

In certain embodiments, the oral formulation comprises a chimeric immunogen and one or more compounds that can protect the chimeric immunogen while it is in the stomach. For example, the protective compound should be able to prevent acid and/or enzymatic hydrolysis of the chimeric immunogen. In certain embodiments, the oral formulation comprises a chimeric immunogen and one or more compounds that can facilitate transit of the immunogen from the stomach to the small intestine. In certain embodiments, the one or more compounds that can protect the chimeric immunogen from degradation in the stomach can also facilitate transit of the immunogen from the stomach to the small intestine. Preferably, the oral formulation comprises one or more compounds that can protect the chimeric immunogen from degradation in the stomach and facilitate transit of the immunogen from the stomach to the small intestine. For example, inclusion of sodium bicarbonate can be useful in facilitating the rapid movement of intra-gastric delivered materials from the stomach to the duodenum as described in Mrsny et al., 1999, Vaccine 17:1425-1433.

Other methods for formulating compositions so that the chimeric immunogens can pass through the stomach and contact polarized epithelial membranes in the small intestine include, but are not limited to, enteric-coating technologies as described in DeYoung, 1989, Int J Pancreatol. 5 Suppl:31-6, and the methods provided in U.S. Pat. Nos. 6,613,332, 6,174,529, 6,086,918, 5,922,680, and 5,807,832.

5.7.2. Dosage

Generally, a pharmaceutically effective amount of the compositions of the invention is administered to a subject. The skilled artisan can readily determine if the dosage of the composition is sufficient to elicit an immune response by monitoring the immune response so elicited, as described below. In certain embodiments, an amount of composition corresponding to between about 1 μg and about 1000 μg of chimeric immunogen is administered. In other embodiments, an amount of composition corresponding to between about 10 μg and about 500 μg of chimeric immunogen is administered. In still other embodiments, an amount of composition corresponding to between about 10 μg and about 250 μg of chimeric immunogen is administered. In yet other embodiments, an amount of composition corresponding to between about 10 μg and about 100 μg of chimeric immunogen is administered. Preferably, an amount of composition corresponding to between about 10 μg and about 50 μg of chimeric immunogen is administered. Further guidance on selecting an effective dose of the compositions may be found, for example, in Rose and Friedman, 1980, Manual of Clinical Immunology, American Society for Microbiology, Washington, D.C.

The volume of composition administered will generally depend on the concentration of chimeric immunogen and the formulation of the composition. In certain embodiments, a unit dose of the composition is between about 0.05 ml and about 1 ml, preferably about 0.5 ml. The compositions can be prepared in dosage forms containing between 1 and 50 doses (e.g., 0.5 ml to 25 ml), more usually between 1 and 10 doses (e.g., 0.5 ml to 5 ml)

The compositions of the invention can be administered in one dose or in multiple doses. A dose can be followed by one or more doses spaced by about 4 to about 8 weeks, by about 1 to about 3 months, or by about 1 to about 6 months. Additional booster doses can be administered as needed. In certain embodiments, booster doses are administered in about 1 to about 10 years.

5.7.3. Administration of Compositions

The compositions of the invention can be administered to a subject by any method known to one of skill in the art. In certain embodiments, the compositions are contacted to a mucosal membrane of the subject. In other embodiments, the compositions are injected into the subject. By selecting one of these methods of administering the compositions, a skilled artisan can modulate the immune response that is elicited. These methods are described extensively below.

Thus, in certain embodiments, the compositions are contacted to a mucosal membrane of a subject. Any mucosal membrane known by one of skill in the art, without limitation, can be the target of such administration. For example, the mucosal membrane can be present in the eye, nose, mouth, lungs, esophagus, stomach, small intestine, large intestine, rectum, anus, vagina, or penis of the subject. Preferably, the mucosal membrane is a nasal mucous membrane or an intestinal mucous membrane.

In other embodiments, the composition is delivered by injection. The composition can be injected subcutaneously or intramuscularly. In such embodiments, the composition preferably comprises an adjuvant, as described above.

5.7.4. Kits Comprising Compositions

In yet another aspect, the invention provides a kit comprising a composition of the invention in one or more sterile containers, e.g., vials. In certain embodiments, the kit further comprises instructions directing a medical professional to administer the composition to a mucous membrane of a subject. In certain embodiments, the kit further comprises instructions directing a medical professional to administer the composition by injection to a subject.

In still another aspect, the present invention provides a kit comprising packaging material and a pharmaceutical composition of the invention contained within the packaging material, said pharmaceutical composition in a form suitable for administration to a subject, preferably a human, or in a format that can be diluted or reconstituted for administration to the subject. In one embodiment, the article of manufacture further comprises printed instructions and/or a label directing the use or administration of the pharmaceutical composition. The instructions and/or label can, for example, suggest a dosing regimen for induction of an immune response against one or more heterologous antigens. Thus, instructions and/or label can provide informational material that advises the physician, technician or subject on how to appropriately induce, monitor, and optionally boost with repeated administration an immune response induced against one or more heterologous antigens.

As with any pharmaceutical product, the packaging material and container of the kits of the invention are designed to protect the stability of the product during storage and shipment. More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical composition of the invention contained within said packaging material.

### 5.8. Making and Testing the Chimeric Immunogens

The chimeric immunogens of the invention are preferably produced recombinantly, as described below, However, the chimeric immunogens may also be produced by chemical synthesis using methods known to those of skill in the art. Alternatively, the chimeric immunogens can be produced using a combination of recombinant and synthetic methods.

5.8.1. Manufacture of Chimeric Immunogens

Methods for expressing and purifying the chimeric immunogens of the invention are described extensively in the examples below. Generally, the methods comprise introducing an expression vector encoding the chimeric immunogen into a cell that can express the chimeric immunogen from the vector. The chimeric immunogen can then be purified for administration to a subject following expression of the immunogen.

5.8.2. Verification of Chimeric Immunogens

Having selected the domains of the chimeric immunogen, the function of these domains, and of the chimeric immunogens as a whole, can routinely be tested to ensure that the immunogens can induce the desired immune response. For example, the chimeric immunogens can be tested for cell recognition, cytosolic translocation and immunogenicity using routine assays. The entire chimeric protein can be tested, or, the function of various domains can be tested by substituting them for native domains of the wild-type toxin.

5.8.2.1. Receptor Binding/Cell Recognition

Receptor binding domain function can be tested by monitoring the chimeric immunogen's ability to bind to the target receptor. Such testing can be accomplished using cell-based assays, with the target receptor present on a cell surface, or in cell-free assays. For example, chimeric immunogen binding to a target can be assessed with affinity chromatography. The chimera can be attached to a matrix in an affinity column, and binding of the receptor to the matrix detected, or vice versa. Alternatively, if antibodies have been identified that bind to either the receptor binding domain or its cognate receptor, the antibodies can be used, for example, to detect the receptor binding domain in the chimeric immunogen by immunoassay, or in a competition assay for the cognate receptor. An exemplary cell-based assay that detects chimeric immunogen binding to receptors on cells comprises labeling the chimera and detecting its binding to cells by, e.g., fluorescent cell sorting, autoradiography, etc.

5.8.2.2. Translocation

The function of the translocation domain can be tested as a function of the chimeric immunogen's ability to gain access to the interior of a cell. Because access first requires binding to the cell, these assays can also be used to assess the function of the cell recognition domain.

The chimeric immunogen's ability to enter the cell can be assessed, for example, by detecting the physical presence of the chimera in the interior of the cell. For example, the chimeric immunogen can be labeled with, for example, a fluorescent marker, and the chimeric immunogen exposed to the cell. Then, the cells can be washed, removing any chimeric immunogen that has not entered the cell, and the amount of label remaining determined. Detecting the label in this fraction indicates that the chimeric immunogen has entered the cell.

5.8.2.3. ER Retention and Translocation to the Cytosol

A related assay can be used to assess the ability of the chimeric immunogen to traffic to the ER and from there into the cytosol of a cell. In such assays, the chimeric immunogen can be labeled with, for example, a fluorescent marker, and the chimeric immunogen exposed to the cell. The cells can then be washed and treated to liberate the cellular contents. The cytosolic fraction of this preparation can then be isolated and assayed for the presence of the label. Detecting the label in this fraction indicates that the chimeric immunogen has entered the cytosol.

In another method, the ability of the translocation domain and ER retention domain to effect translocation to the cytosol can be tested with a construct containing a domain III having ADP ribosylation activity. Briefly, cells expressing a receptor to which the construct binds are seeded in tissue culture plates and exposed to the chimeric protein or to an engineered PE exotoxin containing the modified translocation domain or ER retention sequence in place of the native domains. ADP ribosylation activity can be determined as a function of inhibition of protein synthesis by, e.g., monitoring the incorporation of 3H-leucine.

5.8.2.4. Immunogenicity

The ability of the chimeric immunogens to elicit an immune response against at least one of the heterologous antigens can be assessed by determining the chimeric immunogen's immunogenicity. Humoral, cell-mediated, and secretory immunogenicity can be assessed. For example, a humoral immune response can tested by inoculating an animal with the chimeric immunogen and detecting the production of antibodies specific for at least one of the heterologous antigens with a suitable immunoassay. Such detection is well within the ordinary skill of those in the art. Similarly, a secretory immune response can be tested by detecting in a secreted fluid, for example, saliva, antibodies specific for at least one of the heterologous antigens with a suitable immunoassay.

In addition, cell-mediated immunogenicity can be tested by immunizing an animal with the chimeric immunogen, isolating cytotoxic T cells from the animal, and detecting their ability to kill cells whose MHC Class I molecules bear peptides sharing amino acid sequences with at least one of the heterologous antigens. This assay can also be used to test the activity of the cell recognition domain, the translocation domain and the ER retention domain because generation of a cell mediated response requires binding of the chimera to the cell, trafficking to the ER, and translocation to the cytosol.

The following examples merely illustrate the invention, and are not intended to limit the invention in any way.

### 6. EXAMPLES

### 6.1. Construction of a Chimeric Immunogen

A chimeric immunogen expression vector is generated in a multistep process. A DNA oligonucleotide duplex encoding one of the desired antigens is digested with appropriate restriction enzymes and gel purified (Qiagen Inc., Valencia, Calif.). Alternatively, larger DNA fragment encoding an antigen of interest is prepared from a previously-obtained source, such as a recombinant plasmid or other cloning vehicle. The same procedures are used to purify a DNA fragment encoding another antigen of interest. A DNA fragment of PE encoding amino acids 1-360 is generated by PCR using pPE64pSTΔ553 as a template. See Hertle et al., 2001, Infect. Immun. 69(15): 6962-6969. The PCR fragment is digested with appropriate restriction enzymes and gel purified (Qiagen Inc., Valencia, Calif.). The purified fragments encoding the two or more heterologous antigens of interest and PCR-fragment are ligated into an appropriate site of pPE64pSTΔ553 (i.e., the region encoding domain Ib and/or domain III) depending on the restriction enzymes used to prepare the DNA fragments encoding the antigens and the immune response desired to be induced with a chimeric immunogen expressed from the construct. The final construct is verified by restriction enzyme digestion.

In addition, a toxic form of this chimera is constructed by ligating the antigens of interest together with DNA fragments derived from pPE64-PstI. Such constructs are verified by restriction enzyme digestion. Chimeras expressed from this plasmid are useful as positive controls to assess toxicity of the chimeric immunogen.

### 6.2. Expression of a Chimeric Immunogen

E. coli DH5α cells (Gibco/BRL) are transformed using a standard heat-shock method in the presence of the appropriate plasmid. Transformed cells, selected on antibiotic-containing media, are isolated and grown in Luria-Bertani broth (Difco; Becton Dickinson, Franklin Lakes, N.J.) with antibiotic and induced for protein expression by the addition of 1 mM isopropyl-D-thiogalactopyranoside (IPTG). Two hours following IPTG induction, cells are harvested by centrifugation at 5000 rpm. Inclusion bodies are isolated following cell lysis and proteins are solubilized in 6M guanidine HCl and 2 mM EDTA (pH 8.0) plus 65 mM dithioerythreitol. Following refolding and purification, as previously described (Buchner et al., 1992, Anal. Biochem. 205:263-70; Hertle et al., 2001, Infect. Immun. 69(15): 6962-6969), proteins are stored in PBS (pH 7.4) lacking Ca²⁺ and Mg²⁺ at −80° C.

### 6.3. Characterization of a Chimeric Immunogen

The chimeric immunogen ntPEpilinPAK is prepared by genetically grafting the antigens of interest into domain Ib and/or domain II of ntPE (FIG. 2) as described above. Purified proteins used in these studies are assessed by size-exclusion chromatography using a ZORBAX® GF-450 column (Agilent Technologies, Palo Alto, Calif.) and demonstrated to be greater than 95% monomeric. Additionally, purified chimeric immunogens used in the experiments described herein are determined to have the anticipated mass and composition using amino acid analysis and SDS-PAGE, the correct N-terminal sequence, about 6.5 ng host cell protein/mg chimeric immunogen, <2 pg host cell DNA/mg chimeric immunogen, and about 6.3 EU endotoxin/mg chimeric immunogen.

Cytotoxicity due to inhibition of protein synthesis is examined by exposing L929 (ATCC CCL-1) cells to PE as described previously. See Ogata et al., 1990, J. Biol. Chem. 265:20678-85. Incubation of PE-sensitive L929 cells with either PE or a toxic form of the chimeric immunogen produced as described above result in similar toxicity profiles. This assay is also used to demonstrate a lack of cytotoxicity by the non-toxic form of the chimeric immunogen.

### 6.4. Chimeric Immunogene Immune Response Assays

6.4.1. Isolation of Secreted Antibodies

Mouse saliva (typically 50-100 μl) from mice administered a chimeric immunogen is collected over a 10 min period using a polypropylene Pasteur pipette following the induction of hyper-salivation by an intra-peritoneal injection of 0.1 mg pilocarpine per animal. Serum samples (100 μl) are obtained using serum separators with blood collected from periorbital bleeds. Serum and saliva samples are then aliquoted in 10 μl volumes and stored at −70° C. until analysis. Secreted antibodies thus obtained were characterized in the assays described below.

6.4.2. ELISA Assays

Antibodies against one or more antigens present in a chimeric immunogen are measured by enzyme-linked immunosorbent assay (ELISA). Costar 9018 E.I.A./R.I.A. 96-well plates are coated overnight with 0.6 μg/well of the chimeric immunogen that is used to induce production of the assayed antibodies in 0.2M NaHCO₃-Na₂CO₃, pH 9.4. Each 96-well plate is washed four times with PBS containing 0.05% Tween 20-0.01% thimerosal (wash buffer); and then blocked for 1 h with PBS/Tween 20 containing 0.5% BSA-0.01% thimerosal (assay buffer). Serum and saliva samples are diluted with assay buffer, loaded onto a 96-well plate, and incubated for 2 h for serum IgG and overnight for saliva and serum IgA. Each 96-well plate is then washed four times with wash buffer, and horseradish peroxidase (“HRP”) conjugated goat anti-mouse serum IgG (Pierce Chemical Company, Rockford, Ill.), to assess humoral immune responses, or serum IgA (Kirkegaard & Perry Laboratories, Gaithersburg, Md.), to assess secretory immune responses, is added, then the plates are incubated for 1 and 4 h, respectively. All incubation and coating steps are performed at room temperature covered with parafilm on a shaker at 4 rpm for the specified times. TMB (3,3′,5,5′tetramethylbenzidine), substrate for HRP, is used to quantify bound antibody at 450 nm.

6.4.3. Cell-Mediated Cell Killing Assays

The following examples describe methods that can be used to assess cell-killing by effector cells of the immune system (e.g., cytotoxic T lymphocytes, natural killer cells, etc.) following induction of a cell-mediated immune response with a chimeric immunogen of the invention.

6.4.3.1. Chromium 51 Release Assay

First, effector cells are isolated by standard PBMC isolation procedures. For one NK assay, generally 5-10 mL of whole blood are required, or 5×10⁶ isolated PBMC. K562 cells (human chronic myelogenous leukemia, ATCC #CCL-243) are used as target cells. Other cells can be used as target cells depending on the nature of the antigen used to induce the cell-mediated immune response if the ability to specifically recognize and kill cells that express that antigen is to be tested. For example, in the case of pathogen-derived antigens, cells infected with the pathogen or that express the antigen are used as target cells. In the case of, for example, cancer antigens, cells that express the antigen can be used as target cells, or, preferably, cancer cells that express the antigen are used as target cells.

Complete medium (CM): for culture of K562 target cells, preparation of the effector cells and all assay procedures, RPMI 1640 media supplemented with 2% L-glutamine, 1% penicillin/streptomycin, 10% heat-inactivated fetal calf serum and 2.5% Hepes buffer is used. (All reagents can be obtained from Gibco, Gaithersburg, Md. or equivalent.) Complete medium should be warmed to 37° C. before use in all procedures described below. Sodium Chromate, Na₂⁵¹CRO₄ (⁵¹Cr), is obtained from, e.g., NEN, Dupont, Boston, Mass. or Amersham Life Sciences, Arlington, Ill. The concentration is adjusted to 1 mCi/mL in sterile PBS. Magnetic beads, e.g., Dynabeads M-450, are first coated with sheep anti-mouse IgG1- and coated with anti-CD3 (e.g., (cat. nos. 110.03 and cat. no. M111.13; Dynal, Oslo, Norway). CD56 monoclonal antibody can be obtained from, e.g., NCAM, clone 123C3—cat. no. 18-0152; Zymed Laboratories, CA.

On the day prior to the assay, 1-3×10⁶ K562 cells are placed into a flask with fresh CM. On the day of the assay, the log phase K562 cells are pelleted and resuspended in 1 mL of CM. 1×10⁶ K562 cells per three samples are removed and placed in a fresh 15 mL tube. The cells are then repelleted, and 100 mCi of ⁵¹Cr is added per 1×10⁶ K562 cells, followed by 10% v/v of fetal bovine serum (FBS). The K562 cells are incubated for 1 h at 37° C., shaking the tube every 15 min to resuspend the cells and ensure uniform labeling. After incubation, 10 mL CM is added, the cells are pelleted and gently resuspended. This step is repeated two more times to wash the cells free of excess 51 Cr. After the final wash, the cells are resuspended in 1 mL of CM, counted using a hemacytometer, the concentration of cells is adjusted to 5×10⁴ viable K562 cells/mL.

PBMC (or other suitable effector cells) are separated from whole blood using standard separation techniques, and 5×10⁵ PBMC are resuspended in 5 mL of CM. In order to partially purify the PBMC population, adherent macrophages can be removed by placing the PBMC suspension in a 25 cm² tissue culture flask and incubating the cells at least 1 h at 37° C. The PBMC are then collected and dispensed into a 15 mL centrifuge tube, pelleted, and resuspended in 500 μL of CM. The PBMC are then counted, and the concentration of the cells is adjusted to 5×10⁶ cells/mL. Stepwise dilutions of the PBMC are performed in CM medium to generate aliquots of cells at 2.5, 1.25, and 0.25×10⁶, respectively for the required E:T ratios.

In a 96-well, U-bottomed microtiter plate, 100 μL of each effector cell concentration is dispensed in triplicate. 100 μL of target cells, adjusted to 5×10⁴ cells/mL, is dispensed to every well containing effector cells. For controls, 100 μL of sterile 10% SDS is used to lyse 100 μL of target cells to release all the ⁵¹Cr from the target cells to calculate the maximum release (max), while 100 μL of CM is added to 100 μL of target cells in order to calculate the amount of CR⁵¹ spontaneously released. The cells are then incubated for 4 h at 37° C.

The plate is removed from the incubator and the supernatant fluid is harvested. An aliquot (usually 35 μL) is collected from the 96-well plate using a multichannel pipet and transferred to another 96-well plate in which dry scintillant is coated. After drying the plate overnight, radioactivity is measured in a 96-well format liquid scintillation counter (Packard, CT, USA).

The level of activity as denoted by the percentage specific lysis (% lysis) of labeled targets is determined by the following formula.

$$\% lysis = \frac{mean test c p m - mean spon c p m}{mean max c p m - mean spon c p m} \times 100$$

cpm=counts per minute (mean cpm is usually average of three replicates); test cpm=cpm released by the target cells in the presence of effector cells; spon=cpm released by the target cells in the absence of any effector cells; and max=cpm released by the target cells in the presence of SDS.

In appropriate assays, to verify that the lysis of K562 is NK cell-mediated, specific cell types are depleted from the isolated PBMCs and the change in % lysis against K562 cells is examined. For example, T-cells or NK cells can be depleted from the PBMC, and run the three populations concurrently in the standard NK cell assay. To do so, PBMCs are resuspended at 5×10⁵/mL as described above. The PBMCs are divided equally into three polypropylene tubes with at least 5×10⁶ PBMC/tube, then pelleted and resuspended. One tube is the control depletion, another is for T-cell depletion, and the third is for NK cell depletion. The T-cells can be depleted using, e.g., anti-CD3 monoclonal antibody, while NK cells can be depleted using, e.g., anti-CD56 monoclonal antibody. 20 μL diluted antibody is added per 10⁶ cells, then incubated for 45 min at 4° C., shaking occasionally. The cells are then washed twice with cold PBS with calcium and magnesium. Appropriate anti-mouse (or other species) magnetic beads (e.g., Dynabeads M-450 coated with sheep anti-mouse IgG1-cat. no. 110.03) are added sufficient to yield a bead:cell ratio of 10:1. 3 mL PBS is added, and the solution is placed on a magnet (e.g., Dynal MPC1 or equivalent) for 2 min. The cells are then resuspended in 100 μL media, incubated at 4° C. for 20 min with occasional shaking. These steps are then repeated twice.

Most gamma and liquid scintillation counters can be programmed to calculate the mean epm and percentage specific lysis values (% lysis). Triplicate cpm values should have a mean SEM of less than 5% and values outside of means should be outliered using standard statistical methods. The % lysis values alone can suffice as a measure of NK (or other cell) activity or non-parametric tests, such as Kruskall-Wallis or Wilcoxon tests can be used to determine significant statistical differences between slopes of response curves at different E:T ratios and different groups of patients and controls NK cytotoxic activity can also be expressed as lytic units (LU). An LU is defined as the number of lymphocytes required to yield a particular % lysis, e.g., the LU₂₀ is the number of lytic units that yield 20% lysis of targets by a particular number of effector cells. The % specific lysis is plotted versus the log of the effector cell number for each E:T ratio (for example, in the standard NK set up described above, at the 100:1 ratio there are 5×10⁵ cells per well, at 50:1 there are 2.5×10⁵ effector cells per well, etc.). An NK specific response, for example, is defined as >50% decrease in % specific lysis at two or more E:T ratios in the NK depleted PBMC fraction relative to the whole PBMC. There must be <10% decrease in % specific lysis in the anti-CD3 depleted PBMC relative to the whole PBMC.

6.4.3.2. Flow Cytometry Assay

This example provides an assay that can be used to test the ability of NK cells, cytotoxic T lymphocytes, etc. to kill target cells. As with the Chromium 51 release assay, the target cell used depends on the type of cell-mediated immune response being tested.

The isolation of PBMC from buffy coat is performed as described by Schober et al., 1984, Exp. Cell. Res. 152:348-356. NK cells are isolated using, e.g., the MACS-device and the NK-cell-isolation kit 465-2 (Miltenyi Biotec, Bergisch Gladbach, Germany) according to the manufacturer's recommendations. Membrane staining is performed as follows: a stock solution is prepared by dissolving DIOC18 (Sigma) in DMSO (2 mg/ml; Sigma) over night with agitation. The NK cells (10⁶ cells/ml) are incubated in 10 μg/ml DIOC18 (final concentration) for 1 h at 37° C. Cells are washed twice and maintained in medium (RPMI 1640 [Bio Whittaker, Boehringer Ingelheim, Germany] supplemented with 120% bovine serum and L-glutamine).

K562 target cells and all other cell lines are obtained from the ATCC and kept under aseptic conditions in a 5% CO₂-enriched atmosphere in medium. The stained NK cells are incubated with native target cells at different E/T ratios (1:1; 5:1; 10:1; 20:1), whereby the concentration of effector cells is always 10⁶/ml. Samples are taken at the indicated time points and 5 μg/ml (final concentration) 7-AAD (Sigma) is added. The suspension is analyzed using, e.g., a Coulter Epics XL flow cytometer (Coulter, Krefeld, Germany). The scatter gate is set to all cellular events (including dead cells), and the percentage of vital versus necrotic effector and target cells is calculated from an FL1 (DIOC18) versus FL4 (7-AAD) dot-plot statistic. If additional anti-CD34 surface antibodies are used, these antibodies (e.g., clone HPCA-2; Becton-Dickinson, Hamburg, Germany) are added 15 min before the final analysis and analyzed in the FL2 (PE) channel. The events from the scatter plot were transferred to an FL1 (DIOC18) histogram, and the DIOC18-negative target cells were then transferred to an FL2 (CD34-PE) versus FL4 (7-AAD) plot to calculate the vitality of all cells as well as the CD34-positive target cells specifically.

All experiments are performed in parallel without effector cells. Such background values (typical below 2%) are subtracted from those obtained with effector cells.

6.4.4. Antibody Dependent Cell Killing Assays

Antibody-dependent cell killing assays against one or more of the antigens of the chimeric immunogens are assessed according to the following protocol.

6.4.4.1. Complement-Dependent cytotoxicity.

Cell lysis with baby rabbit complement is determined using a ⁵¹Cr-release assay. Cancer cells bearing an antigen of interest or cells infected with a pathogen from which an antigen of interest is derived are labeled with 0.1 mCi ⁵¹Cr-sodium chromate (New England Nuclear) at 37° C. for 1 hour. The cells are then washed three times with RPMI 1640 medium. ⁵¹Cr-labeled cells (1×10⁴ cells) are incubated with various concentrations of antibody obtained as described above or control IgG on ice for 30 minutes. The unbound antibody is removed by washing the cells three times with medium. The cells are then distributed into 96-well plates and incubated with serial dilutions of baby rabbit complement (Cedarlane, Ontario, Canada) at 37° C. for 2 hours. After incubation, supernatants from each well (50 μL) are harvested and ⁵¹Cr is measured using a gamma counter. Spontaneous release of ⁵¹ Cr is measured after incubating ⁵¹Cr-labeled cells with medium alone. The maximum release of ⁵¹Cr is determined after incubation of ⁵¹ Cr-labeled cells with 1% NP-40. Percentage of cytotoxicity is calculated from the formula: specific cytotoxicity (%)=(A−C)/(B−C)×100, where A=experimental ⁵¹Cr release, B=maximum ⁵¹Cr release, and C=spontaneous ⁵¹Cr release.

6.4.4.2. Antibody-Dependent Cell-Mediated Cytotoxicity (ADCC).

ADCC activity is determined by standard 4-hour ⁵¹Cr-release assay. Splenic mononuclear cells from SCID mice are used as effector cells and cultured in RPMI 1640 medium with or without 500 U/mL of recombinant mouse interleukin (IL)-2 (Genzyme, Cambridge, Mass.) for 6 days, then washed, and resuspended in medium before use. ⁵¹Cr-labeled target cells expressing an antigen of interest, as described above, are placed in 96-well plates and various concentrations of antibody obtained as described above or control IgG are added to wells. Effector cells are then added to the plates at various effector to target (E/T) ratios. After 4 hours incubation, supernatants are removed and counted in a gamma counter. The percentage of cell lysis is determined as above.

6.4.4.3. Statistical Analysis.

The statistical significance in the data of in vitro experiments is determined by the unpaired t-test. The significant differences in survival data are evaluated using a log-rank test.

### 6.5. Vaccination Using a Chimeric Immunogen

8/group BALB/c mice (Charles River Laboratories, Wilmington, Mass.), 6-8 weeks at initial dosing, are used in these studies since age-related suppression of immune function has been demonstrated in this species. See Linton & Dorshkind, 2004, Nat. Immunol. 5:133-9. Intranasal inoculation is performed to mice lightly anesthetized with isoflurane. All intranasal (IN) administrations are performed under mild anesthesia since fluid introduced into the nares of awake mice that is in excess of its cavity volume is rapidly ingested while suppression of this reflex occurs under anesthesia. Thus, administration to anesthetized mice results in preferential delivery to the trachea rather than the esophagus following IN administration. See Janakova et al., 2002, Infect. Immun. 70:5479-84. Mice receive 10 μl of ntPE-pilin (5 μl/nares) in PBS for each immunization. Variations in concentration from 100 μg/ml to 10 mg/ml are prepared for dosing studies to assess immune responses over the range of 1 to 100 μg of chimeric immunogen.

Mice receiving an IN inoculation dose schedule of 0, 7, 14, and 28 days with 1, 10 or 100 μg chimeric immunogen are evaluated for mucosal and systemic humoral immune responses, with similar IN delivery of PBS to mice serving as a negative control. Animals receiving a subcutaneous (SubQ) injection of 10 μg chimeric immunogen in a standard protocol using Freund's complete/incomplete adjuvant materials serve as a positive control.

Immune responses induced by the chimeric immunogen are assessed by detecting salivary (secretory immune response) and serum (humoral immune response) antibodies specific for one or more of the antigens present in the chimeric immunogen. Exemplary methods for detecting such antibodies are described above.

The present invention provides, inter alia, chimeric immunogens and methods of inducing an immune response in a subject. While many specific examples have been provided, the above description is intended to illustrate rather than limit the invention. Many variations of the invention will become apparent to those skilled in the art upon review of this specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. Citation of these documents is not an admission that any particular reference is “prior art” to this invention.

What is claimed is:

1. A chimeric immunogen, comprising:
a)—a cell surface receptor binding domain,
b)—an exotoxin translocation domain, and
c)—more than one non-contiguous heterologous antigen.

2. The chimeric immunogen of claim 1, wherein said cell surface receptor binding domain is selected from the group consisting of domain Ia of Pseudomonas exotoxin A; a receptor binding domains from cholera toxin, diptheria toxin, shiga toxin, or shiga-like toxin; a monoclonal antibody, a polyclonal antibody, or a single-chain antibody; TGFα, TGFβ, EGF, PDGF, IGF, or FGF; IL-1, IL-2, IL-3, or IL-6; and MIP-1α, MIP-1b, MCAF, or IL-8.

3. The chimeric immunogen of claim 2, wherein said cell surface receptor binding domain is domain Ia of Pseudomonas aeruginosa exotoxin A.

4. The chimeric immunogen of claim 3, wherein said domain Ia of Pseudomonas aeruginosa exotoxin A has an amino acid sequence that is SEQ ID NO.: 1.

5. The chimeric immunogen of claim 1, wherein said exotoxin translocation domain is selected from the group consisting of domain II of Pseudomonas aeruginosa exotoxin A, diptheria toxin, pertussis toxin, cholera toxin, heat-labile E. coli enterotoxin, shiga toxin, and shiga-like toxin.

6. The chimeric immunogen of claim 5, wherein said exotoxin translocation domain is domain II of Pseudomonas aeruginosa exotoxin A.

7. The chimeric immunogen of claim 6, wherein said domain II of Pseudomonas aeruginosa exotoxin A has an amino acid sequence that is SEQ ID NO.: 2.

8. The chimeric immunogen of claim 1, wherein at least one of said antigens is connected with said exotoxin translocation domain or said cell surface receptor binding domain with a covalent bond.

9. The chimeric immunogen of claim 8, wherein said covalent bond is a peptide bond.

10. The chimeric immunogen of claim 1, wherein at least one of said antigens is from a pathogen.

11. The chimeric immunogen of claim 1, wherein at least one of said antigens is from a cancer cell.

12. The chimeric immunogen of claim 1, wherein at least two of said antigens are from the same antigenic molecule.

13. The chimeric immunogen of claim 1, wherein none of said antigens is from the same antigenic molecule.

14. The chimeric immunogen of claim 1, wherein at least one of said antigens is a B cell antigen.

15. The chimeric immunogen of claim 1, wherein at least one of said antigens is a T cell antigen.

16. The chimeric immunogen of claim 1, wherein at least one of said antigens is a B cell antigen and at least one of said antigens is a T cell antigen.

17. The chimeric immunogen of claim 1, wherein at least one of said antigens is a peptide or polypeptide antigen.

18. The chimeric immunogen of claim 14, wherein at least two of said antigens are from the same peptide or polypeptide.

19. The chimeric immunogen of claim 14, wherein none of said antigens is from the same peptide or polypeptide.

20. The chimeric immunogen of claim 14, wherein all of said antigens are peptide or polypeptide antigens.

21. The chimeric immunogen of claim 20, wherein at least two of said antigens are from the same peptide or polypeptide.

22. The chimeric immunogen of claim 20, wherein none of said antigens is from the same peptide or polypeptide.

23. The chimeric immunogen of claim 1, wherein said chimeric immunogen further comprises at least a portion of domain Ib of Pseudomonas aeruginosa exotoxin A and at least one of said antigens is inserted into said domain Ib.

24. The chimeric immunogen of claim 23, wherein said antigen that is inserted into domain Ib of Pseudomonas aeruginosa exotoxin A is the V3 loop of HIV gp120 protein.

25. The chimeric immunogen of claim 24, wherein said V3 loop of HIV gp120 protein has an amino acid sequence that is SEQ ID NO.:3.

26. The chimeric immunogen of claim 1, wherein said chimeric immunogen further comprises at least a portion of an enzymatically inactive domain III of Pseudomonas aeruginosa exotoxin A and at least one of said antigens is inserted into said domain III.

27. The chimeric immunogen of claim 26, wherein said antigen inserted into domain III is a T cell antigen.

28. The chimeric immunogen of claim 26, wherein said Domain III of Pseudomonas aeruginosa exotoxin A comprises an endoplasmic reticulum retention signal.

29. The chimeric immunogen of claim 26, wherein said antigen that is inserted into said portion of an enzymatically inactive Domain III of Pseudomonas aeruginosa exotoxin A is nef protein of HIV.

30. The chimeric immunogen of claim 29, wherein said nef protein of HIV has an amino acid sequence that is SEQ ID NO.:4.

31. The chimeric immunogen of claim 24, wherein said chimeric immunogen further comprises at least a portion of an enzymatically inactive Domain III of Pseudomonas aeruginosa exotoxin A, at least one of said antigens is inserted into said Domain III, wherein said antigen that is inserted into said portion of Domain III of Pseudomonas aeruginosa exotoxin A is nef protein of HIV.

32. The chimeric immunogen of claim 29, wherein said V3 loop of HIV gp120 protein has an amino acid sequence that is SEQ ID NO.:3 and said nef protein of HIV has an amino acid sequence that is SEQ ID NO.:4.

33. A polynucleotide encoding a chimeric immunogen according to claim 1.

34. An expression vector that comprises said polynucleotide of claim 33 operably linked to a promoter.

35. The expression vector of claim 34, wherein said promoter is a eukaryotic promoter.

36. The expression vector of claim 34, wherein said promoter is a prokaryotic promoter.

37. The expression vector of claim 34, wherein said promoter is an inducible promoter.

38. The expression vector of claim 34, wherein said expression vector further comprises a secretion signal that directs secretion of a polypeptide expressed from said expression vector from the cell in which said polypeptide is expressed.

39. A transformed or transfected cell that comprises the expression vector of claim 34.

40. A composition comprising a chimeric immunogen according to claim 1.

41. The composition of claim 40, wherein said composition further comprises a pharmaceutically acceptable diluent, excipient, vehicle, or carrier.

42. The composition of claim 41, wherein said composition is formulated for nasal or oral administration.

43. A kit comprising the claim 41, wherein said compositions is in a single-unit dosage form.

44. The kit of claim 43, further comprising instructions directing administration of said composition to a subject.

45. A method for inducing an immune response in a subject, said method comprising contacting an apical epithelial membrane of said subject with an effective amount of a chimeric immunogen according to claim 1.

46. The method of claim 45, wherein said immune response is a humoral immune response.

47. The method of claim 45, wherein said immune response is a secretory immune response.

48. The method of claim 45, wherein said immune response is a cell-mediated immune response.

49. The method of claim 45, wherein said chimeric immunogen is administered in the form of a pharmaceutical composition, wherein said pharmaceutical composition comprises said chimeric immunogen and a pharmaceutically acceptable diluent, excipient, vehicle, or carrier.

50. The method of claim 49, wherein said pharmaceutical composition is formulated for nasal or oral administration.

51. The method of claim 50, wherein said chimeric immunogen is administered to said subject nasally or orally.

52. The method of claim 40, wherein said subject is a mammal.

53. The method of claim 52, wherein said subject is a rodent, lagomorph or primate.

54. The method of claim 53, wherein said subject is a human.
